SEQUENCE LISTING (7 sequences)

SEQ ID NO: 1 - DNA, 21 bp, Artificial
  CD45 PMO targeting translational start site
ccacaaacccatggtcatatc

SEQ ID NO: 2 - DNA, 22 bp, Artificial
  Scrambled sequence part of SC PMO.
cggacacacaaaaagaaagaag

SEQ ID NO: 3 - DNA, 23 bp, Artificial
  Sequence based from CD45 PMO targeting the translational start site
tacgacttaccgatggtgttatc

SEQ ID NO: 4 - DNA, 21 bp, Artificial
  Sequence based from CD45 PMO targeting the translational start site.
ccacaaacccatggtcatatc

SEQ ID NO: 5 - DNA, 15 bp, Artificial
  Sequence based from CD45 PMO targeting the translational start site.
acaaacccatggtca

SEQ ID NO: 6 - DNA, 21 bp, Artificial
  Sequence based from CD45 PMO targeting the translational start site.
cgtgcatgggcaccagtatta

SEQ ID NO: 7 - DNA, 21 bp, Artificial
  Sequence based from CD45 PMO targeting the translational start site.
aacgtttgggtaccagtatat

(19) United States
(12) Patent Application Publication
(10) Pub. No.: US 2012/0135013 A1
(43) Pub. Date: May 31, 2012

(54) CD45 and Methods and Compounds Related Thereto

(76) Inventors: Rekha Panchal, Frederick, MD (US); Sina Bavari, Frederick, MD (US)

(21) Appl. No.: 12/013,648

(22) Filed: Jan. 14, 2008

Related U.S. Application Data
(60) Provisional application No. 60/884,931, filed on Jan. 15, 2007.

(51) Int. Cl. A61K 31/122; A61P 37/04; A61P 31/14; A61P 31/12; A61P 31/04; A61K 31/165; C12N 5/0786

(52) U.S. Cl. 424/184.1; 435/375

(57) ABSTRACT

Disclosed herein are compounds, compositions and methods for preventing, reducing or inhibiting an amount of protein tyrosine phosphatase receptor type C (CD45) expressed or activity of CD45 in a cell or a subject. Also disclosed are methods for preventing, inhibiting or treating an infection in a cell or a subject immunizing a subject or enhancing a subject's immune response against an infection preventing, reducing or inhibiting the susceptibility of a cell or a subject to an infection or subsequent pathogenesis and morbidity due to the infection and preventing, reducing, and inhibiting apoptosis caused by or resulting from a biological agent in a cell or a subject which comprises preventing, reducing or inhibiting an amount of protein tyrosine phosphatase receptor type C (CD45) expressed or activity of CD45 in the cell or the subject.

### CROSS-REFERENCE TO RELATED APPLICATIONS

This application claims the benefit of U.S. Provisional Patent Application Ser. No. 60/884,931, filed 15 Jan. 2007, which is herein incorporated by reference.

### ACKNOWLEDGEMENT OF GOVERNMENT SUPPORT

The present invention was made by employees of the U.S. Government and was made with Government support from Defense Threat Reduction Agency and under contract N01-CO-12400, awarded by National Cancer Institute, National Institutes of Health. The Government has certain rights in this invention.

### BACKGROUND OF THE INVENTION

1. Field of the Invention

The present invention generally relates to treatments for infections which treatments involve CD45.

2. Description of the Related Art

Since 9-11 and the 2001 anthrax attacks, the threat of bioterrorism is real. Biological agents that are considered likely candidates for weaponization, or have been weaponized include Bacillus anthracis, Filoviridae spp., Marburg virus, Yersinia pestis, Vibrio cholerae, Francisella tularensis, Brucella spp., Coxiella burnetii, Arenavirus spp., Coccidioides immitis, Coccidioides posadasii, Burkholderia spp., Shigella spp., Rickettsia spp., Chlamydophila psittaci, Flaviviridae spp., Bunyaviridae, and Variola spp.

Since these biological agents are biologically and genetically diverse, the treatment methods are likewise diverse. For example, anthrax is caused by Bacillus anthracis. Treatment for anthrax infection includes large doses of intravenous and oral antibiotics such as ciprofloxacin, doxycycline, erythromycin, vancomycin and penicillin which must be administered early after infection. Antibiotic therapy will not be effective against antibiotic resistant strains. The anthrax vaccine, BIOTHRAX® requires annual booster injections after the primary injections.

The Ebola viruses (EBOV) are filoviruses (viruses belonging to Filoviridae) associated with outbreaks of highly lethal hemorrhagic fever in humans and primates in North America, Europe, and Africa. Treatment for Ebola hemorrhagic fever is mainly supportive and includes minimizing invasive procedures, balancing electrolytes, preventing and stopping bleeding, maintaining oxygen and blood levels, and treating any complicating infections. Vaccines against EBOV have been developed, but require administration near the time of infection to be effective.

### SUMMARY OF THE INVENTION

The present invention provides methods of preventing, reducing or inhibiting an amount of protein tyrosine phosphatase receptor type C (CD45) expressed or activity of CD45 in a cell or a subject which comprises administering to the cell or the subject an effective amount of

(a) a compound having one of the following structural formulas

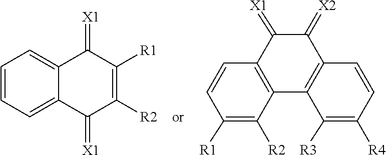

wherein R1, R2, R3, and R4 are each independently selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, an amide possessing an alkyl substituent, or X—R wherein X is O, S, or N and R is selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, or an amide possessing an alkyl substituent; and

X1 and X2 are each independently selected from the group consisting of O, S, or N which may be optionally substituted;

(b) an antisense phosphosphorodiamidate morpholino oligomer (PMO) targeted to CD45; or

(c) knocking down or knocking out the expression or activity of CD45.

In some embodiments, the compound is NSC 148596, NSC 135880, NSC 95397, NSC 270011 or NSC 270012. In some embodiments, the antisense PMO comprises a sequence of at least 15 nucleotide bases and contains at least 15 nucleotide bases which are identical to the nucleotide bases at the corresponding positions of SEQ ID NO:1 or the complement thereof.

In some embodiments, the present invention provides methods of preventing, inhibiting or treating an infection in a cell or a subject which comprises preventing, reducing or inhibiting an amount of protein tyrosine phosphatase receptor type C (CD45) expressed or activity of CD45 in the cell or the subject. In some embodiments, the amount of protein tyrosine phosphatase receptor type C (CD45) expressed or activity of CD45 is prevented, reduced or inhibited by administering to the cell or the subject an effective amount of

(a) a compound having one of the following structural formulas

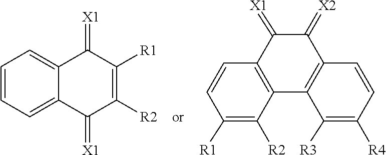

wherein R1, R2, R3, and R4 are each independently selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, an amide possessing an alkyl substituent, or X—R wherein X is O, S, or N and R is selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, or an amide possessing an alkyl substituent; and

X1 and X2 are each independently selected from the group consisting of O, S, or N which may be optionally substituted;

(b) an antisense phosphosphorodiamidate morpholino oligomer (PMO) targeted to CD45; or

(c) knocking down or knocking out the expression or activity of CD45.

In some embodiments, the compound is NSC 148596, NSC 135880, NSC 95397, NSC 270011 or NSC 270012. In some embodiments, the antisense PMO comprises a sequence of at least 15 nucleotide bases and contains at least 15 nucleotide bases which are identical to the nucleotide bases at the corresponding positions of SEQ ID NO:1 or the complement thereof. In some embodiments, the infection is caused by a microorganism or a virus. In some embodiments, the microorganism is a bacterium. In some embodiments, the bacterium is Bacillus anthracis. In some embodiments, the virus belongs to the family Filoviridae. In some embodiments, the virus is an Ebolavirus or a Marburgvirus.

In some embodiments, the present invention provides methods of immunizing a subject or enhancing a subject's immune response against an infection which comprises preventing, reducing or inhibiting an amount of protein tyrosine phosphatase receptor type C (CD45) expressed or activity of CD45 in the cell or the subject. In some embodiments, the amount of protein tyrosine phosphatase receptor type C (CD45) expressed or activity of CD45 is prevented, reduced or inhibited by administering to the cell or the subject an effective amount of

(a) a compound having one of the following structural formulas

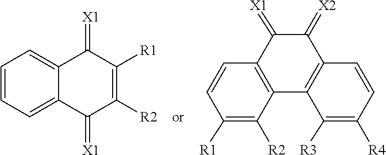

wherein R1, R2, R3, and R4 are each independently selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, an amide possessing an alkyl substituent, or X—R wherein X is O, S, or N and R is selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, or an amide possessing an alkyl substituent; and

X1 and X2 are each independently selected from the group consisting of O, S, or N which may be optionally substituted;

(b) an antisense phosphosphorodiamidate morpholino oligomer (PMO) targeted to CD45; or

(c) knocking down or knocking out the expression or activity of CD45.

In some embodiments, the compound is NSC 148596, NSC 135880, NSC 95397, NSC 270011 or NSC 270012. In some embodiments, the antisense PMO comprises a sequence of at least 15 nucleotide bases and contains at least 15 nucleotide bases which are identical to the nucleotide bases at the corresponding positions of SEQ ID NO:1 or the complement thereof. In some embodiments, the infection is caused by a microorganism or a virus. In some embodiments, the microorganism is a bacterium. In some embodiments, the bacterium is Bacillus anthracis. In some embodiments, the virus belongs to the family Filoviridae. In some embodiments, the virus is an Ebolavirus or a Marburgvirus.

In some embodiments, the present invention provides methods of preventing, reducing or inhibiting the susceptibility of a cell or a subject to an infection or subsequent pathogenesis and morbidity due to the infection which comprises preventing, reducing or inhibiting an amount of protein tyrosine phosphatase receptor type C (CD45) expressed or activity of CD45 in the cell or the subject. In some embodiments, the amount of protein tyrosine phosphatase receptor type C (CD45) expressed or activity of CD45 is prevented, reduced or inhibited by administering to the cell or the subject an effective amount of

(a) a compound having one of the following structural formulas

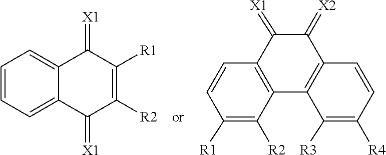

wherein R1, R2, R3, and R4 are each independently selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, an amide possessing an alkyl substituent, or X—R wherein X is O, S, or N and R is selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, or an amide possessing an alkyl substituent; and

X1 and X2 are each independently selected from the group consisting of O, S, or N which may be optionally substituted;

(b) an antisense phosphosphorodiamidate morpholino oligomer (PMO) targeted to CD45; or

(c) knocking down or knocking out the expression or activity of CD45.

In some embodiments, the compound is NSC 148596, NSC 135880, NSC 95397, NSC 270011 or NSC 270012. In some embodiments, the antisense PMO comprises a sequence of at least 15 nucleotide bases and contains at least 15 nucleotide bases which are identical to the nucleotide bases at the corresponding positions of SEQ ID NO:1 or the complement thereof. In some embodiments, the infection is caused by a microorganism or a virus. In some embodiments, the microorganism is a bacterium. In some embodiments, the bacterium is Bacillus anthracis. In some embodiments, the virus belongs to the family Filoviridae. In some embodiments, the virus is an Ebolavirus or a Marburgvirus.

The present invention also provides methods for increasing, improving or enhancing
clearance of a biological agent in a cell or a subject,
an immunological response to a biological agent by a cell or a subject,
the viability of a cell or a subject exposed to or infected with a biological agent, or
the number of macrophages and dendritic cells in a subject infected with a biological agent,
   which comprises preventing, reducing or inhibiting an amount of protein tyrosine phosphatase receptor type C (CD45) expressed or activity of CD45 in the cell or the subject. In some embodiments, the amount of protein tyrosine phosphatase receptor type C (CD45) expressed or activity of CD45 is prevented, reduced or inhibited by administering to the cell or the subject an effective amount of

(a) a compound having one of the following structural formulas

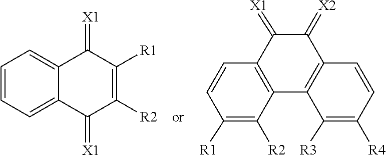

wherein R1, R2, R3, and R4 are each independently selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, an amide possessing an alkyl substituent, or X—R wherein X is O, S, or N and R is selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, or an amide possessing an alkyl substituent; and

X1 and X2 are each independently selected from the group consisting of O, S, or N which may be optionally substituted;

(b) an antisense phosphosphorodiamidate morpholino oligomer (PMO) targeted to CD45; or

(c) knocking down or knocking out the expression or activity of CD45.

In some embodiments, the compound is NSC 148596, NSC 135880, NSC 95397, NSC 270011 or NSC 270012. In some embodiments, the antisense PMO comprises a sequence of at least 15 nucleotide bases and contains at least 15 nucleotide bases which are identical to the nucleotide bases at the corresponding positions of SEQ ID NO:1 or the complement thereof. In some embodiments, the biological agent is a microorganism or a virus. In some embodiments, the microorganism is a bacterium. In some embodiments, the bacterium is Bacillus anthracis. In some embodiments, the virus belongs to the family Filoviridae. In some embodiments, the virus is an Ebolavirus or a Marburgvirus.

The present invention also provides methods for preventing, reducing, or inhibiting apoptosis caused by or resulting from a biological agent in a cell or a subject, which comprises preventing, reducing or inhibiting an amount of protein tyrosine phosphatase receptor type C (CD45) expressed or activity of CD45 in the cell or the subject. In some embodiments, the amount of protein tyrosine phosphatase receptor type C (CD45) expressed or activity of CD45 is prevented, reduced or inhibited by administering to the cell or the subject an effective amount of

(a) a compound having one of the following structural formulas

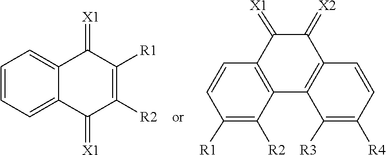

wherein R1, R2, R3, and R4 are each independently selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, an amide possessing an alkyl substituent, or X—R wherein X is O, S, or N and R is selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, or an amide possessing an alkyl substituent; and

X1 and X2 are each independently selected from the group consisting of O, S, or N which may be optionally substituted;

(b) an antisense phosphosphorodiamidate morpholino oligomer (PMO) targeted to CD45; or

(c) knocking down or knocking out the expression or activity of CD45.

In some embodiments, the compound is NSC 148596, NSC 135880, NSC 95397, NSC 270011 or NSC 270012. In some embodiments, the antisense PMO comprises a sequence of at least 15 nucleotide bases and contains at least 15 nucleotide bases which are identical to the nucleotide bases at the corresponding positions of SEQ ID NO:1 or the complement thereof. In some embodiments, the biological agent is a microorganism or a virus. In some embodiments, the microorganism is a bacterium. In some embodiments, the bacterium is Bacillus anthracis. In some embodiments, the virus belongs to the family Filoviridae. In some embodiments, the virus is an Ebolavirus or a Marburgvirus.

In some embodiments, the present invention provides an isolated oligonucleotide comprising a sequence of at least 15 nucleotide bases and contains at least 15 nucleotide bases which are identical to the nucleotide bases at the corresponding positions of SEQ ID NO:1 or the complement thereof. In some embodiments, the nucleotide bases are contiguous.

In some embodiments, the present invention provides methods of preventing, inhibiting or treating an infection caused by Bacillus anthracis or a virus belonging to the family Filoviridae, such as Ebolavirus or a Marburgvirus, in a cell or a subject which comprises preventing, reducing or inhibiting an amount of protein tyrosine phosphatase receptor type C (CD45) expressed or activity of CD45 in the cell or the subject.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description, serve to explain the principles of the invention.

### DESCRIPTION OF THE DRAWINGS

This invention is further understood by reference to the drawings wherein:

FIG. 1 shows that NSC 148596, NSC 135880, NSC 95397, NSC 270011 and NSC 270012 protected macrophages from anthrax lethal toxin (LT) induced cytotoxicity with ED₅₀ values ranging from 5 to 25 μM.

FIG. 2 shows that neither NSC 95397 nor NSC 270012 inhibited proteasome activity compared to MG132, a known proteasome inhibitor.

FIG. 3A shows that NSC 95397 and NSC 270012 protected macrophages after infection with Sterne B. anthracis spores in vitro. The percentage of live and dead cells is indicated. Data from a typical experiment, which was repeated three times with similar results, is shown.

FIG. 3B shows that NSC 95397 and NSC 270012 did not exhibit any significant anti-microbial activity.

FIG. 3C is a table showing that NSC 95397 (10 μM) demonstrated potent in vitro inhibition of CD45 phosphatase activity when screened against a panel of sixteen different phosphatases.

FIG. 4A shows gene targeted knock-down of CD45 in J774A.1 cells that were untreated or incubated with 8 μM of CD45 PMO (CD 45) or scrambled PMO (SC).

FIG. 4B shows an immunoblot of protein lysates prepared from J774A.1 cells that were untreated (0) or treated with CD45 PMO (CD 45) or scrambled PMO (SC) for 72 hours. Reduced levels of CD45 were observed in macrophages treated with CD45 PMO.

FIG. 4C shows a concomitant reduction in CD45 phosphatase activity following immunoprecipitation of CD45 from protein lysates that were either untreated (Un) or treated with CD45 PMO (CD45) or SC PMO. Average data from three independent experiments is shown and ±s.d.

FIG. 5 shows that J774A.1 cells treated with CD45 PMO showed increased viability against infection with Sterne B. anthracis spores in a dose dependent manner when compared to the untreated cells or cells treated with scrambled PMO.

FIG. 6 is an immunoblot of cell lysates of J774A.1 cells untreated and treated with CD45 PMO (CD45 PMO) or SC PMO which shows that MEK cleavage was not prevented in these cells when infected with Sterne (S) B. anthracis spores.

FIG. 7A shows that survival was greatly increased in the animals treated with CD45 PMO following infection with Ames B. anthracis spores. In contrast animals treated with PBS or Scrambled PMO (SC) control did not survive B. anthracis challenge.

FIG. 7B is a table showing that mice treated with CD45 PMO and survived B. anthracis infection developed protective antigen specific and lethal toxin neutralizing antibody titers and were completely protected when re-challenged with Ames B. anthracis spores.

FIG. 8 shows that mice expressing reduced levels of CD45 (CD45^{11%} mice, CD45^{36%} mice and CD45^{62%} mice) when challenged with Ames B. anthracis spores showed increased survival compared to CD45^{100%}, CD45^{0%} or CSV10 +/− (CSV10^{62%}) mice with inactive CD45 phosphatase activity. Mice were challenged via intraperitoneal (i.p.) route with ˜500 cfu of Ames strain of B. anthracis.

FIG. 9 shows immunohistochemical stains of spleen tissues did not show any bacterial load in the CD45^{62%} mice surviving infection with B. anthracis after 48 hours versus moribund CD45^{100%} mice (48 hours). The left panel of FIG. 9 is spleen tissue samples from a CD45^{100%} mouse and the right panel is spleen tissue sample from a CD45^{62%} mouse which were stained with anti-capsule antibody. Scale bar=100 μm, 20× magnification.

FIG. 10 shows that mice expressing different levels of CD45 (CD45^{22%} mice, CD45^{36%} mice, CD45^{62%} mice, CD45^{77%} mice and CD45^{100%} mice) and vaccinated with anthrax vaccine adsorbed (AVA) all generated similar levels of anti-PA specific antibodies compared to the control mice. The different shaped labels correspond to individual mouse in each group.

FIG. 11A is a table showing the genotypes of the transgenic and heterozygous mice and corresponding % CD45 expression.

FIG. 11B shows CD45 expression levels of transgenic and heterozygous mice. Peritoneal macrophages and cells were isolated from spleen or lymph node from the CD45^{100%}, CD45^{62%}, CD45^{36%}, CD45^{22%}, and CD45^{11%} mice.

FIG. 12A shows that reduced CD45 expression does not affect the ability of macrophages to internalize the Sterne B. anthracis spores. The data represents averages from three independent experiments±standard deviation (s.d.).

FIG. 12B shows that reduced CD45 expression does not affect the ability of the macrophages to kill the bacteria. The data represents averages from three independent experiments±standard deviation (s.d.).

FIG. 13 shows that reduced CD45 levels may regulate, reduce, or inhibit apoptosis in thioglycolate elicited peritoneal macrophages. The data represents averages of three independent experiments±standard error (s.e).

FIG. 14A shows that splenocytes isolated from mice post B. anthracis challenge indicated a significant increase in the percentage of CD11b⁺ CD11c⁻ macrophages (24 hours), Ly6G⁺ granulocytes (42 hours), CD8⁺ CD44^{high+} T cells (0, 6 and 42 hours) and CD4⁺ CD44^{high+} T cells (6 and 24 hours) in CD45^{62%} mice. The data represent the averages of four mice/group/time points±standard error (s.e).

FIG. 14B shows that blood samples collected from mice euthanized at time 0, 6, 24 and 42 hours post B. anthracis infection exhibit an increased percentage of Ly6G⁺ granulocytes (6 and 24 hours) and CD8⁺ CD44^{high+} T cells (0, 24 hours) in CD45^{62%} mice. The data represent the averages of four mice/group/time point and ±s.e.

FIG. 15A shows that mice having reduced levels of CD45 expression (CD45^{11%} mice, CD45^{22%} mice, CD45^{36%} mice, CD45^{62%} mice, CD45^{77%} mice) are protected following challenge with EBOV. In contrast, CD45^{100%}, CD45^{77%} or CSV10 +/+ (CSV10^{100%}) mice or CSV10 +/− (CSV10^{62%}) mice that have no phosphatase activity did not survive EBOV challenge.

FIG. 15B shows immunohistochemical stains of spleen tissue from moribund CD45^{100%} mice (left panels) and CD45^{62%} mice surviving EBOV challenge (right panels). Specifically, FIG. 15B shows immunohistochemical tissue stains to detect viral load in the spleen of a moribund CD45^{100%} mouse (day 7, left panel) and a CD45^{62%} mouse (right panel) surviving EBOV challenge (30 day post challenge). Scale bar=100 μm, 4× magnification (top panels); scale bar=100 μm, 20× magnification (bottom panels).

FIG. 16A shows that reduced CD45 expression does not impair humoral immune responses as mice that survived EBOV challenge develop EBOV-specific antibodies.

FIG. 16B shows that reduction in CD45 expression does not affect ex vivo viral replication in splenocytes.

FIG. 17 shows cytokine and chemokine levels from the plasma of CD45^{100%} and CD45^{62%} mice after EBOV infection. The data represent the averages of four mice/group/time points±standard deviation (s.d.).

FIG. 18A shows that splenocytes isolated from mice post EBOV infection exhibited a significant increase in the percentage of CD11b⁺ CD11c⁻ macrophages, Ly6G⁺ granulocytes at day 5 post EBOV challenge and CD8⁺ CD44^{high+} T cells at days 0 and 5 post challenge in the CD45^{62%} mice versus the CD45^{100%} mice. The data represent the averages of four mice/group/time point and ±s.e.

FIG. 18B shows that blood cells collected from mice euthanized at days 0, 1, 3 and 5 post EBOV infection exhibit increased activated CD8⁺ CD44^{high+} T cells (day 0-5 post challenge) in the CD45^{62%} mice as compared to the CD45^{100%} mice. The data represent the averages of four mice/group/time points±s.e.

FIG. 19A shows immunohistochemical staining of liver, spleen and lymph node tissue from day 7 post-EBOV infected CD45^{100%} mouse (top panels) and a CD45^{62%} mouse (bottom panels). Scale bar=100 um, 20× magnification.

FIG. 19B shows immunohistochemical staining of liver, spleen and lymph node tissue from day 10 post-EBOV infected CD45^{62%} mouse. Scale bar=100 um, 20× magnification.

FIG. 20A shows TUNEL staining of spleen from a CD45^{100%} mouse (top panels) and a CD45^{62%} (bottom panels) mouse euthanized at day 5 and day 7 post EBOV infection. Apoptosis was detected by TUNEL assays. Scale bar=100 um, 20× magnification.

FIG. 20B shows TUNEL staining of liver from a CD45^{100%} mouse (top panels) and a CD45^{62%} (bottom panels) mouse euthanized at day 5 and day 7 post EBOV infection. Apoptosis was detected by TUNEL assays. Scale bar=100 um, 20× magnification.

FIG. 21 shows the results of the viral titer plaque assays based on CD45^{100%} and CD45^{62%} mice that were infected with EBOV and euthanized at days 1, 3, 5, 7, 10, 16 and 21. Tissue (kidney, spleen, liver) was harvested and viral titers were determined by traditional plaque assays.

FIG. 22 shows that reduced CD45 expression has an effect on gene expression and EBOV pathogenesis.

Color versions of these Figures may be found on the World Wide Web at 69.89.17.19/˜datacons/sbavari/figures.pdf, which is herein incorporated by reference.

### DETAILED DESCRIPTION OF THE INVENTION

The present invention relates to compositions and methods for preventing, modulating, reducing or inhibiting the expression level of a protein tyrosine phosphatase (PTP) in a cell or a subject. The present invention also relates to methods and compositions for preventing, inhibiting or treating an infection in a cell or a subject which involves preventing, modulating, reducing or inhibiting the amount of a PTP expressed in the cell or the subject. The present invention relates to methods and compositions for preventing, modulating, reducing or inhibiting the susceptibility of a cell or a subject to an infection and subsequent pathogenesis and morbidity caused by biological agents such as viruses, microorganisms and other pathogens, including viruses belonging to the Bornaviridae, Filoviridae, Paramyxoviridae, Rhabdoviridae, Arenaviridae, Bunyaviridae, Orthomyxoviridae, Poxyiridae, Coronaviridae, Flaviviridae, Herpesviridae, Picornaviridae, Retroviridae, Hepadnaviridae, Papovaviridae, Picornaviridae, Togoviridae, Rhabdoviridae, and Arenaviridae families and bacterial agents such as Bacillus anthracis, Yersinia pestis, Francisella tularensis, Burkholderia spp., Mycobacterium spp., and Coxiella burnetii by preventing, modulating, reducing or inhibiting the amount of a PTP expressed in the cell or the subject.

In some embodiments, the PTP is protein tyrosine phosphatase, receptor type C (CD45). CD45 is a family of high molecular weight glycoproteins which is one of the most abundant leukocyte cell surface glycoproteins and includes isoforms thereof. See e.g. Trowbridge & Thomas (1994) Ann. Rev. Immunol. 12: 85-116 and Stanton T., et al. (2004) Immunogenetics 56(2):107-110, which are herein incorporated by reference.

As exemplified herein, when the amount of CD45 was lowered in subjects, the subjects were protected against infection with Bacillus anthracis and Ebola virus (EBOV). Since B. anthracis is a spore-forming gram positive bacterium and EBOV is an enveloped, non-segmented, negative-strand RNA virus belonging to the Filoviridae family of viruses, the present invention is also directed to compositions and methods for preventing, inhibiting or treating infections resulting from biologically and genetically diverse biological agents, e.g. bacterial agents and viral agents.

Although the experiments exemplified herein are based on mice and mouse cells and tissues, other subjects, such as humans, non-human primates, and other animals, and cells and tissues thereof are contemplated herein.

As used herein, “CD45^{11%} mice” refers to mice which express about 11% of CD45 as compared to levels expressed by wild type mice (CD45^{100%} mice). See Virts, E. L. et al. (2003) Blood 101:849-855, and Virts & Raschke (2001) J. Biol. Chem. 276:19913-19920, which are herein incorporated by reference. Similarly, as used herein, “CD45^{22%} mice”, “CD45^{36%} mice”, “CD45^{62%} mice” and “CD45^{77%} mice” refer to mice which express about 22%, 36%, 62%, and 77% CD45 as compared to levels expressed by wild type mice, respectively. As used herein, “CD45^{0%} mice” refers to knockout mice which do not express any observable amount of CD45. As used herein, “CSV10 +/− mice” refer to mice which express 62% CD45 as compared to levels expressed by wild type mice, but do not exhibit any observable CD45 phosphatase activity. Similarly, “CSV10+/+ mice” refer to mice which express about 100% CD45 as compared to levels expressed by wild type mice, but do not exhibit any observable CD45 phosphatase activity due to a mutant CD45 protein.

As disclosed herein, reduced expression levels of CD45 in a cell or a subject increases, enhances or improves the cell or the subject's ability to clear or respond to an infection, e.g. improves or enhances the immune clearance of the infection. Specifically, as exemplified herein, immune clearance of bacterial and viral infections in CD45^{62%} mice was found to be dependent on CD45 phosphatase activity, as CSV10 mice, which do not exhibit any observable CD45 phosphatase activity, were found to be susceptible to infection by both B. anthracis and EBOV and subsequent pathogenesis and morbidity.

In the studies with EBOV, CD45 expression levels at 62% the norm resulted in hyperactivation of CD8⁺T cells. Additionally, 5 days after infection with EBOV, the percentages of splenic granulocytes and macrophages in the CD45^{62%} mice increased, thereby suggesting enhanced cellular trafficking and migration into the tissues. Based on microarray data, there was a controlled host response to EBOV infection in the CD45^{62%} mice as compared to the CD45^{100%} mice. Thus, active homeostasis, enhanced cell trafficking and migration, and control in disease pathogenesis, or a combination thereof may contribute to the increased or enhanced immunity, pathogen clearance and survival in the CD45^{62%} mice.

As disclosed herein, in studies involving B. anthracis, macrophages expressing reduced levels of CD45 showed increased cell survival following infection with B. anthracis, but not when treated with anthrax lethal toxin (LT). This suggests that the bacterium and its virulent factor LT do not exploit the same host target to modulate or disrupt the downstream signaling processes. As provided herein, the observed protection of CD45 PMO treated macrophages following B. anthracis infection did not correlate with mitogen activated protein kinase kinase (MAPKK/MEK) protection as these cells exhibited a MAPKK cleavage pattern similar to the control cells. These results suggest that the relevant CD45 dependent pathway may not signal through or involve MEK. However, as disclosed herein, reduced apoptosis was observed in macrophages obtained from CD45^{62%} mice as compared to CD45^{100%} mice (controls), thereby indicating that reduced expression levels of CD45 may regulate apoptosis caused by or resulting from infection with a pathogen such as B. anthracis.

The in vivo data discussed herein suggest that the increased number of splenic and peripheral macrophages and dendritic cells (DCs) following infection with B. anthracis in CD45^{62%} mice may induce innate and T cell-mediated responses resulting in bacterial clearance. The observed robust immunity is not an inherent condition of genetic reduction of CD45 expression levels, as knockdown of CD45 by PMO in wild type mice had a survival rate similar to the CD45^{62%} mice following infection with B. anthracis.

### Bacterial Infection

To identify compounds that inhibit anthrax lethal toxin-induced cytotoxicity, a library of small molecules from the National Cancer Institute (NCI) were screened using a toxin-induced cell death assay known in the art. See Panchal, R. G. et al. (2007) Chem. Biol. 14:245-255, which is herein incorporated by reference. Specifically, J774A.1 cells were pre-incubated for one hour with either medium containing DMSO (control) or different concentrations (0 μM to 25 μM) of a given compound, and then treated with anthrax lethal toxin (LT; PA 80 ng/ml and LF 16 ng/ml). Cell viability was determined using a MTT dye assay known in the art. Five compounds, NSC 148596, NSC 135880, NSC 95397, NSC 270011 and NSC 270012, were found to inhibit anthrax LT cytotoxicity with ED₅₀ values ranging from about 5 to about 25 μM. See FIG. 1. Table 1 shows the chemical structures of NSC 148596, NSC 135880, NSC 95397, NSC 270011 and NSC 270012, their percentage of Cdc25B phosphatase inhibition at a concentration of 10 μM and their ED₅₀ values.

TABLE 1
Two dimensional chemical representation of small
molecule with percent cdc25B (at compound concentration
of 10 μM) and ED50 values

| | | % | |
|---|---|---|---|
| | NSC | Inhibition | ED50 |
| structure | number | (cdc25B) | (μM) |
| 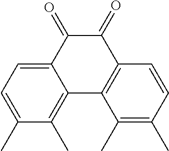 | 135880 | 99 | 25.0 |
| 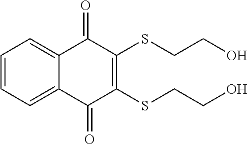 | 95397 | 83 | 6-12 |
| 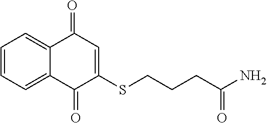 | 270012 | 72 | 6-12 |
| 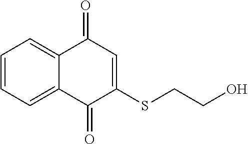 | 148596 | 67 | — |
| 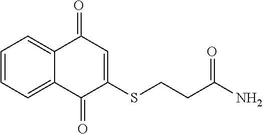 | 270011 | 25 | 6-12 |

The protective effect of NSC 95397 was observed by visualizing the uptake of the membrane-impermeable SYTOX green dye (Invitrogen, Carlsbad, Calif.) by dead cells using time-lapse imaging and methods known in the art. When J774A.1 cells were treated with both NSC 95397 and anthrax LT, relatively few cells took up the dye as compared to those treated with the toxin alone (data not shown), thereby evidencing that NSC 95397 prevented, inhibited or reduced the toxic (lethal) action of anthrax LT on cells.

Therefore, the present invention provides compounds having one of the following structural formulas

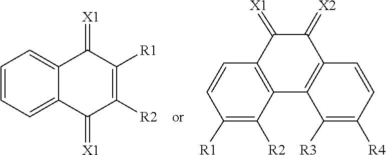

wherein R1, R2, R3, and R4 are each independently selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, an amide possessing an alkyl substituent, or X—R wherein X is O, S, or N and R is selected from the group consisting of hydrogen, amino, amine with stabilized carbocations, carboxyl, optionally substituted alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, or an amide possessing an alkyl substituent; and

X1 and X2 are each independently selected from the group consisting of O, S, or N which may be optionally substituted.

In some embodiments, the compound of the present invention is NSC 148596, NSC 135880, NSC 95397, NSC 270011 or NSC 270012. Compounds and compositions of the present invention also include those provided in U.S. Publication Nos. 20070112048 and 20070112049, which are herein incorporated by reference.

In accordance with a convention used in the art, [symbol] is used in structural formulas herein to depict the bond that is the point of attachment of the moiety or substituent to the core or backbone structure. It is noted that in the structural formulas of the present invention, the bond orders of the specified rings may vary when the various heteroatoms introduce specific requirements to satisfy aromaticity, prevent antiaromaticity, and stabilize tautomeric forms due to localization. Thus, the appropriate bond orders of the ring structures in the structural formulas of the present invention are contemplated herein.

Where chiral carbons are included in chemical structures, unless a particular orientation is depicted, both sterioisomeric forms are intended to be encompassed.

A “halo” or “halogen” means fluorine, bromine, chlorine, and iodine.

An “alkyl” is intended to mean a straight or branched chain monovalent radical of saturated and/or unsaturated carbon atoms and hydrogen atoms, such as methyl (Me), ethyl (Et), propyl (Pr), isopropyl (i-Pr), butyl (n-Bu), isobutyl (i-Bu), t-butyl (t-Bu), (sec-Bu), and the like, which may be unsubstituted (i.e., contain only carbon and hydrogen) or substituted by one or more suitable substituents as defined below. A “lower alkyl group” is intended to mean an alkyl group having from 1 to 8 carbon atoms in its chain.

A “haloalkyl” refers to an alkyl that is substituted with one or more same or different halo atoms, e.g., —CH₂Cl, —CF₃, —CH₂CF₃, —CH₂CCl₃, and the like.

An “alkenyl” means straight and branched hydrocarbon radicals having from 2 to 8 carbon atoms and at least one double bond such as ethenyl, 3-buten-1-yl, 2-ethenylbutyl, 3-hexen-1-yl, and the like. The term “alkenyl” includes, cycloalkenyl, and heteroalkenyl in which 1 to 3 heteroatoms selected from O, S, N or substituted nitrogen may replace carbon atoms.

An “alkynyl” means straight and branched hydrocarbon radicals having from 2 to 8 carbon atoms and at least one triple bond and includes, but is not limited to, ethynyl, 3-butyn-1-yl, propynyl, 2-butyn-1-yl, 3-pentyn-1-yl, and the like.

A “cycloalkyl” is intended to mean a non-aromatic monovalent monocyclic or polycyclic radical having from 3 to 14 carbon atoms, each of which may be saturated or unsaturated, and may be unsubstituted or substituted by one or more suitable substituents as defined herein, and to which may be fused one or more aryl groups, heteroaryl groups, cycloalkyl groups, or heterocycloalkyl groups which themselves may be unsubstituted or substituted by one or more substituents. Examples of cycloalkyl groups include cyclopropyl, cycloheptyl, cyclooctyl, cyclodecyl, cyclobutyl, adamantyl, norpinanyl, decalinyl, norbornyl, cyclohexyl, and cyclopentyl.

A “heterocycloalkyl” is intended to mean a non-aromatic monovalent monocyclic or polycyclic radical having 1-5 heteroatoms selected from nitrogen, oxygen, and sulfur, and may be unsubstituted or substituted by one or more suitable substituents as defined herein, and to which may be fused one or more aryl groups, heteroaryl groups, cycloalkyl groups, or heterocycloalkyl groups which themselves may be unsubstituted or substituted by one or more substituents. Examples of heterocycloalkyl groups include oxiranyl, pyrrolidinyl, piperidyl, tetrahydropyran, and morpholinyl.

An “aryl” (Ar) is intended to mean an aromatic monovalent monocyclic or polycyclic radical comprising generally between 5 and 18 carbon ring members, which may be unsubstituted or substituted by one or more suitable substituents as defined herein, and to which may be fused one or more cycloalkyl groups, heterocycloalkyl groups, or heteroaryl groups, which themselves may be unsubstituted or substituted by one or more suitable substituents. Thus, the term “aryl group” includes a benzyl group (Bzl). Examples include phenyl, biphenyl, 1,2,3,4-tetrahydronaphthyl, naphthyl, anthryl, and phenanthryl.

A “heteroaryl” is intended to mean an aromatic monovalent monocyclic or polycyclic radical comprising generally between 4 and 18 ring members, including 1-5 heteroatoms selected from nitrogen, oxygen, and sulfur, which may be unsubstituted or substituted by one or more suitable substituents as defined below, and to which may be fused one or more cycloalkyl groups, heterocycloalkyl groups, or aryl groups, which themselves may be unsubstituted or substituted by one or more suitable substituents. Examples include thienyl, furanyl, thiazolyl, triazolyl, imidazolyl, isoxazolyl, oxadiazolyl, tetrazolyl, pyridyl, pyrrolyl, thiadiazolyl, oxadiazolyl, oxathiadiazolyl, thiatriazolyl, pyrimidinyl, isoquinolinyl, quinolinyl, napthyridinyl, phthalimidyl, benzimidazolyl, and benzoxazolyl.

A “hydroxy” is intended to mean the radical —OH.

An “alkoxy” is intended to mean the radical —OR, where R is an alkyl group. Exemplary alkoxy groups include methoxy, ethoxy, propoxy, and the like.

A “hydroxyalkyl” means an alkyl that is substituted with one, two, or three hydroxy groups, e.g. hydroxymethyl, 1 or 2-hydroxyethyl, 1,2-, 1,3-, or 2,3-dihydroxypropyl, and the like.

A “haloalkoxy” refers to an —O-(haloalkyl) group. Examples include trifluoromethoxy, tribromomethoxy, and the like.

A “cycloalkoxy” is intended to mean the radical —OR, where R is acycloalkyl or heterocycloalkyl group.

An “aryloxy” is intended to mean the radical —OR, where R is an aryl or heteroaryl group. Examples include phenoxy, pyridinyloxy, furanyloxy, thienyloxy, pyrimidinyloxy, pyrazinyloxy, and the like.

An “acyl” is intended to mean a —C(O)—R radical, where R is an alkyl or aryl, bonded through a carbonyl group. Acyl groups include acetyl, benzoyl, and the like.

An “aralkyl” means an alkyl that is substituted with an aryl group. Examples include —CH₂-phenyl, —(CH₂)₂-phenyl, —(CH₂)₃-phenyl, —CH₃CH(CH₃)CH₂-phenyl, and the like.

A “heteroaralkyl” group means an alkyl that is substituted with a heteroaryl group. Examples include —CH₂-pyridinyl, —(CH₂)₂-pyrimidinyl, —(CH₂)₃-imidazolyl, and the like.

A “carboxy” is intended to mean the radical —C(O)OH.

An “alkoxycarbonyl” is intended to mean the radical —C(O)OR, where R is an alkyl group. Examples include methoxycarbonyl, ethoxycarbonyl, and the like.

An “amino” is intended to mean the radical —NH₂.

An “amine with stabilized carbocations” are comprised of two or more NH₂ groups that contribute lone pairs to configure a highly stabilized carbocation. Examples include amidines and guanidines.

An “alkylamino” is intended to mean the radical —NHR, where R is an alkyl group or the radical —NR^{a}R^{b}, where R^{a} and R^{b} are each independently an alkyl group. Examples of alkylamino groups include methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino, N-n-hexyl-N-methylamino and the like.

An “alkylsulfhydryl” is intended to mean R—SH, where R is an alkyl group. Examples include methylsulfhydryl, ethylsulfhydryl, n-propylsulfhydryl, iso-propylsulfhydryl, n-butylsulfhydryl, iso-butylsulfhydryl, secondary-butylsulfhydryl, tertiary-butylsulfhydryl. Preferable alkylsulfhydryl groups are methylsulfhydryl, ethylsulfhydryl, n-propylsulfhydryl, n-butylsulfhydryl, and the like.

An “alkylhydroxymate” is intended to mean the radical R—C(O)NH—OH, where R is an alkyl group. Examples include methylhydroxymate, ethylhydroxymate, n-propylhydroxymate, iso-propylhydroxymate, n-butylhydroxymate, iso-butylhydroxymate, secondary-butylhydroxymate, tertiary-butylhydroxymate. Preferable alkylhydroxymate groups are methylhydroxymate, ethylhydroxymate, n-propylhydroxymate, n-butylhydroxymate, and the like. A “carbamoyl” is intended to mean the radical —C(O)NH₂.

An “alkylaminocarbonyl” is intended to mean the radical —C(O)NHR, where R is an alkyl group or the radical —C(O)NR^{a}R^{b}, where R^{a} and R^{b} are each independently an alkyl group. Examples include methylaminocarbonyl, ethylaminocarbonyl, dimethylaminocarbonyl, methylethylaminocarbonyl, and the like.

A “mercapto” is intended to mean the radical —SH.

An “alkylthio” is intended to mean the radical —SR, where R is an alkyl or cycloalkyl group. Examples of alkylthio groups include methylthio, ethylthio, n-propylthio, isopropylthio, tert-butylthio, n-pentylthio, n-hexylthio, cyclopropylthio, cyclobutylthio, cyclopentylthio, cyclohexylthio, and the like.

An “arylthio” is intended to mean the radical —SR, where R is an aryl or heteroaryl group. Examples include phenylthio, pyridinylthio, furanylthio, thienylthio, pyrimidinylthio, and the like.

A “thioacyl” is intended to mean a —C(S)—R radical, where R is an alkyl or aryl, bonded through a thiol group.

An “alkylsulfonyl” is intended to mean the radical —SO₂R, where R is an alkyl group. Examples include methylsulfonyl, ethylsulfonyl, n-propylsulfonyl, iso-propylsulfonyl, n-butylsulfonyl, iso-butylsulfonyl, secondary-butylsulfonyl, tertiary-butylsulfonyl. Preferable alkylsulfonyl groups are methylsulfonyl, ethylsulfonyl, n-propylsulfonyl, n-butylsulfonyl, and the like.

A “leaving group” (Lv) is intended to mean any suitable group that will be displaced by a substitution reaction. One of ordinary skill in the art will know that any conjugate base of a strong acid can act as a leaving group. Illustrative examples of suitable leaving groups include, but are not limited to, —F, —Cl, —Br, alkyl chlorides, alkyl bromides, alkyl iodides, alkyl sulfonates, alkyl benzenesulfonates, alkyl p-toluenesulfonates, alkyl methanesulfonates, triflate, and any groups having a bisulfate, methyl sulfate, or sulfonate ion.

A “protecting group” is intended to refer to groups that protect one or more inherent functional group from premature reaction. Suitable protecting groups may be routinely selected by those skilled in the art in light of the functionality and particular chemistry used to construct the compound. Examples of suitable protecting groups are described, for example, in Greene and Wuts, Protective Groups in Organic Synthesis, 3^{rd} edition, John Wiley and Sons, New York, N.Y. (1999).

The term “suitable organic moiety” is intended to mean any organic moiety recognizable, such as by routine testing, to those skilled in the art as not adversely affecting the inhibitory activity of the inventive compounds. Illustrative examples of suitable organic moieties include, but are not limited to, hydroxyl groups, alkyl groups, oxo groups, cycloalkyl groups, heterocycloalkyl groups, aryl groups, heteroaryl groups, acyl groups, sulfonyl groups, mercapto groups, alkylthio groups, alkoxyl groups, carboxyl groups, amino groups, alkylamino groups, dialkylamino groups, carbamoyl groups, arylthio groups, heteroarylthio groups, and the like.

In general, the various moieties or functional groups for variables in the formulae may be “optionally substituted” by one or more suitable “substituents”. The term “substituent” or “suitable substituent” is intended to mean any suitable substituent that may be recognized or selected, such as through routine testing, by those skilled in the art. Illustrative examples of useful substituents are those found in the exemplary compounds that follow, as well as a halogen; C₁₋₆-alkyl; C₁₋₆-alkenyl; C₁₋₆-alkynyl; hydroxyl; C₁₋₆ alkoxyl; amino; nitro; thiol; thioether; imine; cyano; amido; phosphonato; phosphine; carboxyl; carbonyl; aminocarbonyl; thiocarbonyl; sulfonyl; sulfonamine; sulfonamide; ketone; aldehyde; ester; oxygen (═O); haloalkyl (e.g., trifluoromethyl); carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl); carbocyclic or heterocyclic, monocyclic or fused or non-fused polycyclic aryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); nitro; thiol; thioether, O-lower alkyl; O-aryl, aryl; aryl-lower alkyl; CO₂CH₃; CONH₂; OCH₂CONH₂; NH₂; SO₂NH₂; OCHF₂; CF₃; OCF₃; and the like. Such moieties may also be optionally substituted by a fused-ring structure or bridge, for example OCH₂—O. All of these substituents may optionally be further substituted with a substituent selected from groups such as hydroxyl groups, halogens, oxo groups, alkyl groups, acyl groups, sulfonyl groups, mercapto groups, alkylthio groups, alkyloxyl groups, cycloalkyl groups, heterocycloalkyl groups, aryl groups, heteroaryl groups, carboxyl groups, amino groups, alkylamino groups, dialkylamino groups, carbamoyl groups, aryloxyl groups, heteroaryloxyl groups, arylthio groups, heteroarylthio groups, and the like.

The term “optionally substituted” is intended to expressly indicate that the specified group is unsubstituted or substituted by one or more suitable substituents, unless the optional substituents are expressly specified, in which case the term indicates that the group is unsubstituted or substituted with the specified substituents. As defined above, various groups may be unsubstituted or substituted (i.e., they are optionally substituted) unless indicated otherwise herein (e.g., by indicating that the specified group is unsubstituted).

It is understood that while a compound of the general structural formulas herein may exhibit the phenomenon of tautomerism, the structural formulas within this specification expressly depict only one of the possible tautomeric forms. It is therefore to be understood that the structural formulas herein are intended to represent any tautomeric form of the depicted compound and is not to be limited merely to a specific compound form depicted by the structural formulas.

It is also understood that the structural formulas are intended to represent any configurational form of the depicted compound and is not to be limited merely to a specific compound form depicted by the structural formulas.

Some of the compounds of the present invention may exist as single stereoisomers (i.e., essentially free of other stereoisomers), racemates, or mixtures of enantiomers, diastereomers, or both when they contain one or more stereogenic centers as designated by R or S according to the Cahn-Ingold-Prelog rules whether the absolute or relative configuration is known. All such single stereoisomers, racemates and mixtures thereof are intended to be within the scope of the present invention.

Some of the compounds in the present invention may exist as geometric isomers as the result of containing a stereogenic double bond. In such cases, they may exist either as pure or mixtures of cis or trans geometric isomers or (E) and (Z) designated forms according to the Cahn-Ingold-Prelog rules and include compounds that adopt a double bond configuration as a result of electronic delocalization.

As generally understood by those skilled in the art, an optically pure compound having one or more chiral centers (i.e., one asymmetric atom producing unique tetrahedral configuration) is one that consists essentially of one of the two possible enantiomers (i.e., is enantiomerically pure), and an optically pure compound having more than one chiral center is one that is both diastereomerically pure and enantiomerically pure. If the compounds of the present invention are made synthetically, they may be used in a form that is at least 90% optically pure, that is, a form that comprises at least 90% of a single isomer (80% enantiomeric excess (e.e.) or diastereomeric excess (d.e.), more preferably at least 95% (90% e.e. or d.e.), even more preferably at least 97.5% (95% e.e. or d.e.), and most preferably at least 99% (98% e.e. or d.e.).

Additionally, the structural formulas herein are intended to cover, where applicable, solvated as well as unsolvated forms of the compounds. A “solvate” is intended to mean a pharmaceutically acceptable solvate form of a specified compound that retains the biological effectiveness of such compound. Examples of solvates include compounds of the invention in combination with water, isopropanol, ethanol, methanol, dimethyl sulfoxide, ethyl acetate, acetic acid, ethanolamine, or acetone. Also included are miscible formulations of solvate mixtures such as a compound of the invention in combination with an acetone and ethanol mixture. In a preferred embodiment, the solvate includes a compound of the invention in combination with about 20% ethanol and about 80% acetone. Thus, the structural formulas include compounds having the indicated structure, including the hydrated as well as the non-hydrated forms.

As indicated above, the compounds of the invention also include active tautomeric and stereoisomeric forms of the compounds of the present invention, which may be readily obtained using techniques known in the art. For example, optically active (R) and (S) isomers may be prepared via a stereospecific synthesis, e.g., using chiral synthons and chiral reagents, or racemic mixtures may be resolved using conventional techniques.

Additionally, the compounds of the invention include pharmaceutically acceptable salts, multimeric forms, prodrugs, active metabolites, precursors and salts of such metabolites of the compounds of the present invention.

The term “pharmaceutically acceptable salts” refers to salt forms that are pharmacologically acceptable and substantially non-toxic to the subject being treated with the compound of the invention. Pharmaceutically acceptable salts include conventional acid-addition salts or base-addition salts formed from suitable non-toxic organic or inorganic acids or inorganic bases. Exemplary acid-addition salts include those derived from inorganic acids such as hydrochloric acid, hydrobromic acid, hydroiodic acid, sulfuric acid, sulfamic acid, phosphoric acid, and nitric acid, and those derived from organic acids such as p-toluenesulfonic acid, methanesulfonic acid, ethane-disulfonic acid, isethionic acid, oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, and fumaric acid. Exemplary base-addition salts include those derived from ammonium hydroxides (e.g., a quaternary ammonium hydroxide such as tetramethylammonium hydroxide), those derived from inorganic bases such as alkali or alkaline earth-metal (e.g., sodium, potassium, lithium, calcium, or magnesium) hydroxides, and those derived from non-toxic organic bases such as basic amino acids.

The term “multimer” refers to multivalent or multimeric forms of active forms of the compounds of the invention. Such “multimers” may be made by linking or placing multiple copies of an active compound in close proximity to each other, e.g., using a scaffolding provided by a carrier moiety. Multimers of various dimensions (i.e., bearing varying numbers of copies of an active compound) may be tested to arrive at a multimer of optimum size with respect to binding site interactions. Provision of such multivalent forms of active binding compounds with optimal spacing between the binding site moieties may enhance binding site interactions. See e.g. Lee et al., (1984) Biochem. 23:4255, which is herein incorporated by reference. The artisan may control the multivalency and spacing by selection of a suitable carrier moiety or linker units. Useful moieties include molecular supports comprising a multiplicity of functional groups that can be reacted with functional groups associated with the active compounds of the invention. A variety of carrier moieties may be used to build highly active multimers, including proteins such as BSA (bovine serum albumin), peptides such as pentapeptides, decapeptides, pentadecapeptides, and the like, as well as non-biological compounds selected for their beneficial effects on absorbability, transport, and persistence within the target organism. Functional groups on the carrier moiety, such as amino, sulfhydryl, hydroxyl, and alkylamino groups, may be selected to obtain stable linkages to the compounds of the invention, optimal spacing between the immobilized compounds, and optimal biological properties.

“A pharmaceutically acceptable prodrug” is a compound that may be converted under physiological conditions or by solvolysis to the specified compound or to a pharmaceutically acceptable salt of such compound, or a compound that is biologically active with respect to the intended pharmacodynamic effect. “A pharmaceutically active metabolite” is intended to mean a pharmacologically active product produced through metabolism in the body of a specified compound or salt thereof. Prodrugs and active metabolites of a compound may be identified using routine techniques known in the art. See, e.g., Bertolini, G. et al., (1997) J. Med. Chem. 40:2011-2016; Shan, D. et al., J. Pharm. Sci., 86(7):765-767; Bagshawe K., (1995) Drug Dev. Res. 34:220-230; Bodor, N., (1984) Advances in Drug Res. 13:224-331; Bundgaard, H., Design of Prodrugs (Elsevier Press, 1985); and Larsen, I. K., Design and Application of Prodrugs, Drug Design and Development (Krogsgaard-Larsen et al., eds., Harwood Academic Publishers, 1991), which are herein incorporated by reference.

If the compound of the present invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyrvic acid, oxalic acid, glycolic acid, salicylic acid, a pyranosidyl acid, such as glucuronic acid or galacturonic acid, an α-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the present invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from basic amino acids, such as lysine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of compounds that are solids, it is understood by those skilled in the art that the compound of the present invention and salts may exist in different crystal or polymorphic forms, all of which are intended to be within the scope of the present invention and specified structural formulas.

The compounds and compositions of the present invention are useful for preventing, reducing or inhibiting an amount of protein tyrosine phosphatase receptor type C (CD45) expressed or activity of CD45 in a cell or a subject. The activity of the compounds and compositions of the present invention may be measured by any of the methods available to those skilled in the art, including in vitro and in vivo assays. Examples of suitable assays for activity measurements are provided herein. Properties of the compounds of the present invention may be assessed, for example, by using one or more of the assays set out in the Examples below. Thus, one skilled in the art may readily screen, without undue experimentation, a given compound falling within the structural formulas described herein to determine whether it is capable of preventing, reducing or inhibiting an amount of protein tyrosine phosphatase receptor type C (CD45) expressed or activity of CD45 in a cell or a subject and therefore falls within the scope of the instant invention. Other pharmacological methods may also be used to determine the efficacy of the compounds a subject suffering from a given disease or disorder. The compounds of the present invention may be used in combination with or as a substitution for treatments known in the art.

The therapeutically effective amounts of the compounds of the invention for treating the diseases or disorders described above in a subject can be determined in a variety of ways known to those of ordinary skill in the art, e.g. by administering various amounts of a particular compound to a subject afflicted with a particular condition and then determining the effect on the subject. Typically, therapeutically effective amounts of a compound of the present invention can be orally administered daily at a dosage of the active ingredient of 0.002 to 200 mg/kg of body weight. Ordinarily, a dose of 0.01 to 10 mg/kg in divided doses one to four times a day, or in sustained release formulation will be effective in obtaining the desired pharmacological effect. It will be understood, however, that the specific dose levels for any particular subject will depend upon a variety of factors including the activity of the specific compound employed, the age, body weight, general health, sex, diet, time of administration, route of administration, and rate of excretion, drug combination and the severity of the particular disease.

Frequency of dosage may also vary depending on the compound used and the particular disease treated. It will also be appreciated that the effective dosage of the compound used for treatment may increase or decrease over the course of a particular treatment. Changes in dosage may result and become apparent by standard diagnostic assays known in the art. In some instances chronic administration may be required. The compounds of the present invention may be administered before, during, after, or a combination thereof exposure to bacteria.

As provided herein, an “effective amount” is intended to mean that amount of a compound or composition that is sufficient to reduce, prevent or inhibit an amount of protein tyrosine phosphatase receptor type C (CD45) expressed or activity of CD45 in a cell or a subject as compared with a negative control. A “therapeutically effective amount” of a compound or composition of the present invention, is a quantity sufficient to, when administered to a subject, reduce, prevent or inhibit an amount of protein tyrosine phosphatase receptor type C (CD45) expressed or activity of CD45 in the subject. Also, as used herein, a “therapeutically effective amount” of a compound of the present invention is an amount which prevents, inhibits, suppresses, or reduces a given clinical condition in a subject as compared to a control. As defined herein, a therapeutically effective amount of a compound of the present invention may be readily determined by one of ordinary skill by routine methods known in the art.

The pharmaceutical formulations of the invention comprise at least one compound of the present invention and may be prepared in a unit-dosage form appropriate for the desired mode of administration. The pharmaceutical formulations of the present invention may be administered for therapy by any suitable route including oral, rectal, nasal, topical (including buccal and sublingual), dermal, mucosal, vaginal and parenteral (including subcutaneous, intramuscular, intravenous and intradermal). It will be appreciated that the preferred route will vary with the condition and age of the recipient, the nature of the condition to be treated, and the chosen compound of the present invention.

The compound can be administered alone, but will generally be administered as pharmaceutical formulations suitable for administration. Pharmaceutical formulations known in the art contemplated herein. Pharmaceutical formulations of this invention comprise a therapeutically effective amount of at least one compound of the present invention, and an inert, pharmaceutically or cosmetically acceptable carrier or diluent. As used herein the language “pharmaceutically acceptable carrier” or a “cosmetically acceptable carrier” is intended to include any and all solvents, dispersion media, coatings, antibacterial and antifungal agents, isotonic and absorption delaying agents, and the like, compatible with pharmaceutical or cosmetic administration. Except insofar as any conventional media or agent is incompatible with the active compound, use thereof in the formulation is contemplated. Descriptions of suitable pharmaceutically acceptable carriers, formulations, and factors involved in their selection, are found in a variety of readily available sources, e.g., Remington's Pharmaceutical Sciences, 17^{th} ed., Mack Publishing Company, Easton, Pa., 1985, and Remington: The Science and Practice of Pharmacy, 21^{th} ed., Lippincott Williams & Wilkins, 2005, which are incorporated herein by reference.

Supplementary active compounds can also be incorporated into the formulations. Supplementary active compounds include antibiotics, antiviral agents, antiprotozoal agents, antifungal agents, and antiproliferative agents known in the art, analgesics and other compounds commonly used to treat diseases and disorders associated with viral infection and toxic side effects of viral infection.

Antibiotics include penicillin, cloxacillin, dicloxacillin, methicillin, nafcillin, oxacillin, ampicillin, amoxicillin, bacampicillin, azlocillin, carbenicillin, mezlocillin, piperacillin, ticarcillin, azithromycin, clarithromycin, clindamycin, erythromycin, lincomycin, demeclocycline, doxycycline, minocycline, oxytetracycline, tetracycline, quinolone, cinoxacin, nalidixic acid, fluoroquinolone, ciprofloxacin, enoxacin, grepafloxacin, levofloxacin, lomefloxacin, norfloxacin, ofloxacin, sparfloxacin, trovafloxacin, bacitracin, colistin, polymyxin B, sulfonamide, trimethoprim-sulfamethoxazole, co-amoxyclav, cephalothin, cefuroxime, ceftriaxone, vancomycin, gentamicin, amikacin, metronidazole, chloramphenicol, nitrofurantoin, co-trimoxazole, rifampicin, isoniazid, pyrazinamide, kirromycin, thiostrepton, micrococcin, fusidic acid, thiolactomycin, fosmidomycin, and the like.

Antiviral agents include abacavir, aciclovir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, gardasil, ibacitabine, immunovir, idoxuridine, imiquimod, indinavir, inosine, lamivudine, lopinavir, loviride, maraviroc, nelfinavir, nevirapine, nexavir, oseltamivir, penciclovir, peramivir, pleconaril, podophyllotoxin, ribavirin, rimantadine, ritonavir, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, zidovudine, and the like.

Antiprotozoal agents include chloroquine, doxycycline, mefloquine, metronidazole, eplornithine, furazolidone, hydroxychloroquine, iodoquinol, pentamidine, mebendazole, piperazine, halofantrine, primaquine, pyrimethamine sulfadoxine, doxycycline, clindamycin, quinine sulfate, quinidine gluconate, quinine dihydrochloride, hydroxychloroquine sulfate, proguanil, quinine, clindamycin, atovaquone, azithromycin, suramin, melarsoprol, eflornithine, nifurtimox, amphotericin B, sodium stibogluconate, pentamidine isethionate, trimethoprim-sulfamethoxazole, pyrimethamine, sulfadiazine, and the like.

Antifungal agents include amphotericin B, fluconazole, itraconazole, ketoconazole, potassium iodide, flucytosine, and the like.

Antiproliferative agents such as altretamine, amifostine, anastrozole, arsenic trioxide, bexarotene, bleomycin, busulfan, capecitabine, carboplatin, carmustine, celecoxib, chlorambucil, cisplatin, cisplatin-epinephrine gel, cladribine, cytarabine liposomal, daunorubicin liposomal, daunorubicin daunomycin, dexrazoxane, docetaxel, doxorubicin, doxorubicin liposomal, epirubicin, estramustine, etoposide phosphate, etoposide VP-16, exemestane, fludarabine, fluorouracil 5-FU, fulvestrant, gemicitabine, gemtuzumab-ozogamicin, goserelin acetate, hydroxyurea, idarubicin, ifosfamide, imatinib mesylate, irinotecan, letrozole, leucovorin, levamisole, liposomal daunorubicin, melphalan L-PAM, mesna, methotrexate, methoxsalen, mitomycin C, mitoxantrone, paclitaxel, pamidronate, pegademase, pentostain, porfimer sodium, streptozocin, talc, tamoxifen, temozolamide, teniposide VM-26, topotecan, toremifene, tretinoin, ATRA, valrubicin, vinorelbine, zoledronate, steroids, and the like.

Medicaments for preventing, reducing or inhibiting an amount of protein tyrosine phosphatase receptor type C (CD45) expressed or activity of CD45 in a cell or a subject comprising the compounds and compositions of the present invention and methods of manufacturing the medicaments are contemplated herein.

Toxicity and therapeutic efficacy of the compounds and compositions disclosed herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The present invention also provides methods of reducing, inhibiting, or treating the toxicity of anthrax LT a cell or a subject which comprises administering to the cell or the subject an effective amount of a compound of the present invention, such as NSC 148596, NSC 135880, NSC 95397, NSC 270011 or NSC 270012.

To determine if NSC 148596, NSC 135880, NSC 95397, NSC 270011 and NSC 270012 inhibited LF enzymatic activity, all five compounds were tested in vitro using an HPLC-based LF assay known in the art. See Panchal et al. (2004) Nat. Struct. Mol. Biol. 11(1):67-72, which is herein incorporated by reference. None of the compounds were observed to inhibit the ability of LF to cleave peptide substrate (data not shown), thereby suggesting that the compounds protected the cells by acting on a cellular component of the cell.

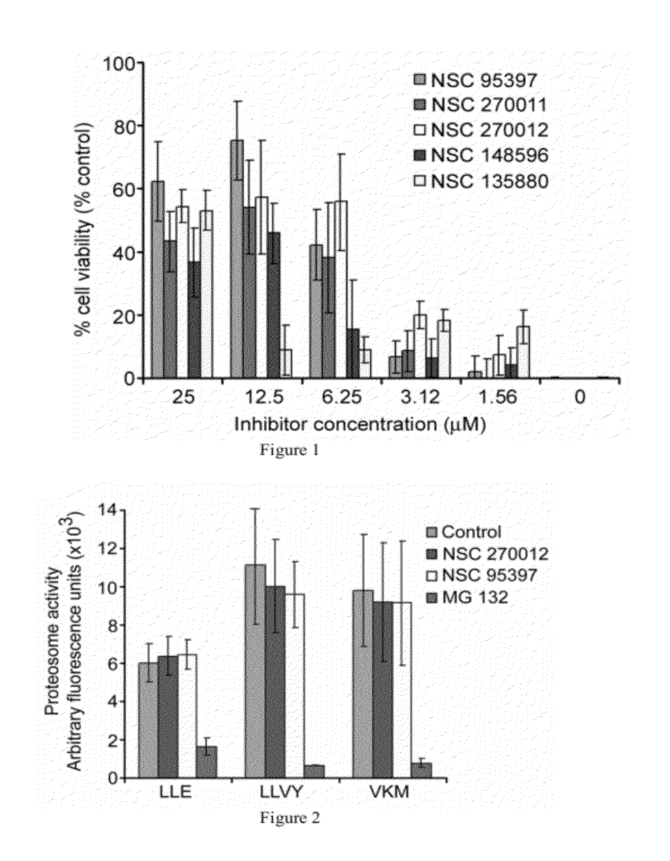

The proteasome, is a large protein complex that causes extra-lysosomal degradation of cellular proteins and plays a role in apoptosis. See Drexler (1997) PNAS USA 94(3):855-860, which is herein incorporated by reference. Functional proteasome activity is reported to be indispensable for anthrax LT to kill macrophage-like cell lines such as RAW264.7. See Tang & Leppla (1999) Infect. Immun. 67(6):3055-3060, which is herein incorporated by reference. To determine if NSC 95397 and NSC 270012 inhibited proteasome activity in J774A.1 macrophages, protein lysates from macrophages were incubated with three different fluorogenic proteasome substrates (LLE, LLVY and VKM). See Panchal, R. G. et al. (2007) Chem. Biol. 14:245-255, which is herein incorporated by reference. As shown in FIG. 2, neither NSC 95397 nor NSC 270012 inhibited proteasome activity as compared to MG132, a known proteasome inhibitor, which suggests that NSC 95397 and NSC 270012 protect against anthrax LT by a mechanism which does not involve proteasome activity.

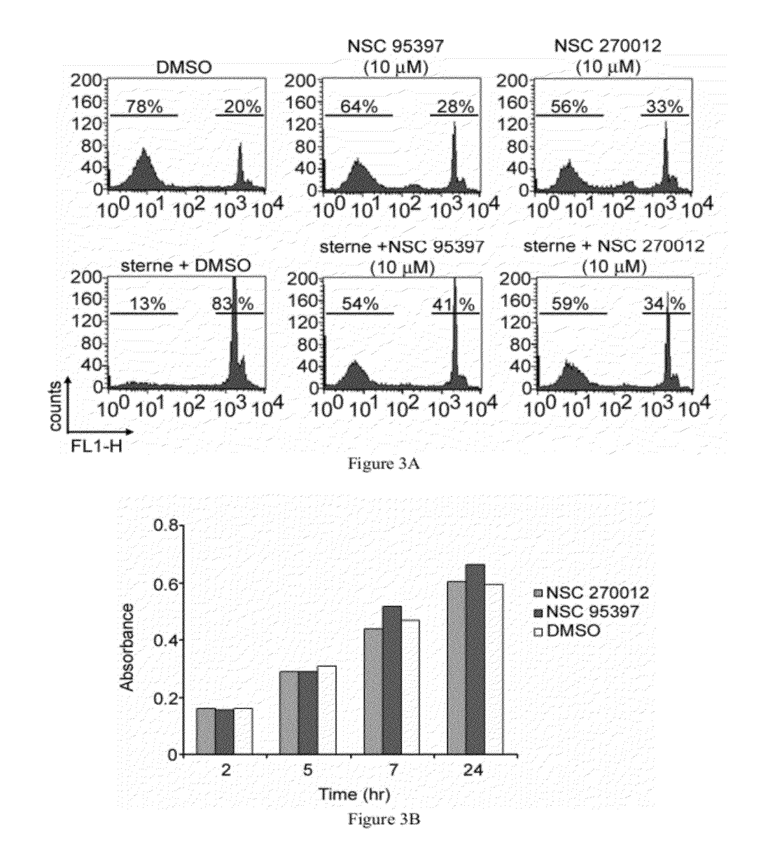

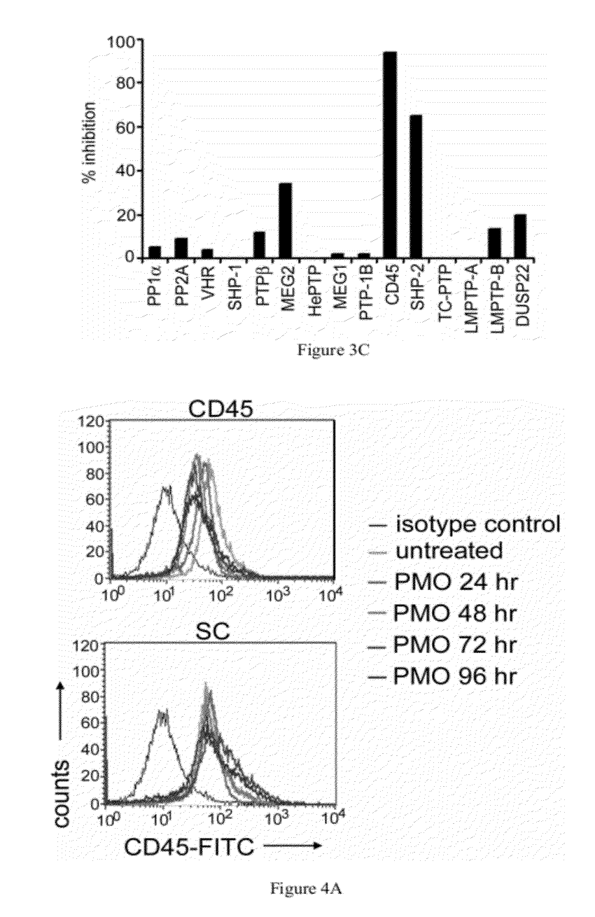

To determine if the compounds of the present invention protect J774A.1 cells against infection with Sterne B. anthracis spores, the cells were pre-incubated for 1 hour with 10 μM of NSC 95397 or NSC 270012 and then incubated with Sterne B. anthracis spores for about 4 hours. Specifically, J774A.1 cells (6×10⁵) were seeded in a volume of 0.5 ml DMEM (Invitrogen, Carlsbad, Calif.) containing 10% fetal bovine serum (FBS, complete medium) in 1.5 ml centrifuge tubes. The cells were pre-incubated for 1 hour at 37° C. with NSC 95397 (10 μM) or NSC 270012 (10 μM) and were subsequently contacted with Sterne B. anthracis spores at multiplicity of infection (MOI) of 5. After incubation for 4 hours at 37° C., bacterial growth was inhibited by the addition of penicillin (100 IU) and streptomycin (100 μg/ml). To determine cell viability, SYTOX green dye (1 μM, Invitrogen, Carlsbad, Calif.) that is impermeable to live cells was added and then the cells were incubated for 15 minutes at 37° C. The cells were centrifuged at 2000 rpm for 2 minutes and then washed two times with complete medium containing penicillin and streptomycin. The cells were fixed with 1% formaldehyde for 15 minutes and then analyzed by flow cytometry methods known in the art. FIG. 3A shows that NSC 95397 and NSC 270012 increased the viability of J774A.1 cells following infection with Sterne B. anthracis spores.

FIG. 3B shows that NSC 95397 and NSC 270012 are not anti-microbial as they do not inhibit the growth of the bacteria. Specifically, Sterne B. anthracis spores were diluted in Mueller-Hinton media (BD Biosciences, San Diego, Calif.) and distributed in a 96-well format at 1×10⁶/100 μl per well. Sterne B. anthracis spores were treated with either DMSO, NSC 95397 (10 μM) or NSC 270012 (10 μM) and at various time intervals bacterial growth was monitored at absorbance of 600 nm.

Because NSC 148596, NSC 135880, NSC 95397, NSC 270011 and NSC 270012 do not directly inhibit anthrax LF enzymatic activity in vitro, it was hypothesized that the compounds target a cellular protein as NSC 95397 and NSC 135880 were previously reported to inhibit Cdc25. See Lazo et al. (2002) Mol. Pharmacol. 61(4):720-728, which is herein incorporated by reference. Cdc25B is a dual-specific phosphatase that regulates entry of all eukaryotic cells into mitosis by activating the cdc2/cyclin B mitotic kinase complex. See Nilsson & Hoffmann (2000) Prog. Cell Cycle Res. 4:107-114, which is herein incorporated by reference. To investigate if Cdc25B was a cellular target involved in anthrax LT-induced cell death, the phosphorylation state of cdc2 and whether G2/M arrest occurred in J774A.1 cells following compound treatment was investigated. Cell lysates from cells treated with the given compounds did not show increased phosphorylation of cdc2 by either Western blotting or immunoprecipitation of the cdc2/cyclin B complex. Further, the treated cells did not exhibit G2/M arrest, although G2/M arrest could be achieved using nocodozole as a positive control (data not shown). These results suggest that Cdc25B is not involved in anthrax LT pathogenesis and morbidity.

After numerous cell-based experiments failed to confirm the role of Cdc25 phosphatases in the anthrax infection model (data not shown), the inhibitory effect NSC 95397 was screened in vitro against a panel of fifteen different phosphatases. FIG. 3C is showing that 10 μM of NSC 95397 demonstrated in vitro inhibition of CD45 phosphatase activity.

In these phosphatase activity experiments, phosphatases purchased from Upstate (Lake Placid, N.Y.) and a generic substrate, DiFMUP (6,8-difluoro-4-methylumbelliferyl phosphate), was (Invitrogen, Carlsbad, Calif.) were used. All assays were performed in 50 mM HEPES containing 1 mM DTT (Sigma-Aldrich, St. Louis, Mo.) and 0.1% BSA (Sigma Aldrich, St. Louis, Mo.), pH 7.4 with the following modifications or additions: SHPT, PTPMEG2, PTPβ, and YopH (10 mM MgCl₂); PP1α and PP2A (10 mM MnCl₂); HePTP, VHR, CD45, TC-PTP, SHP-2, LMPTPA (pH 4.5) and LMPTPB (pH 4.5); PTPMEG-1 (4.8 mM MgCl₂ and 3.2 mM MnCl₂); PTP-1B and DUSP22 (25 mM HEPES, 50 mM NaCl, 5 mM DTT and 2.5 mM EDTA) (Upstate, Lake Placid, N.Y.). NSC 95397 (10 μM) was added to 15 μl of each phosphatase and incubated for 10 minutes and then 10 μl of DiFMUP was added to give a final concentration of 100 μM. The 384 well plate was incubated at room temperature for 60 minutes and then read in an Analyst (MDC using excitation 360 nm; emission 450 nm). The effect of NSC 95397 was compared to control wells containing DMSO. At about 10 μM, NSC 95397 inhibited about 94% of the phosphatase activity of CD45, which is significantly more than the amounts of inhibition on the other phosphatases, thereby suggesting that CD45 may be the cellular target involved in anthrax pathogenesis and morbidity.

To confirm that CD45 is involved in anthrax pathogenesis, a peptide conjugated phosphorodiamidate morpholino oligomer that improves cellular entry and targets the translational start site of CD45 mRNA (CD45 PMO) was synthesized using methods known in the art. See e.g. Moulton et al. (2004) Bioconjug. Chem. 15(2):290-299, which is herein incorporated by reference. Specifically, the sequence of the CD45 PMO targeting the translational start site was 5′ CCACAAACCCATGGTCATATC 3′ (SEQ ID NO:1). The scrambled sequence part of SC PMO, 5′ CGGACACACAAAAAGAAAGAAG 3′ (SEQ ID NO:2), was used as a nonbacterial negative control. For efficient delivery of PMOs into cells, an Arg-rich peptide (CH₃CONH-(RAhxR)₄-Ahx-βAla, designated P007; in which R stands for arginine, Ahx stands for 6-aminohexanoic acid, and βAla stands for beta-alanine) was covalently conjugated to the 5′ end of the PMO through a non-cleavable piperazine linker.

In some embodiments, the antisense PMOs of the present invention comprise a sequence of at least 15 nucleotide bases and contains at least 15 nucleotide bases which are identical to the nucleotide bases at the corresponding positions of SEQ ID NO:1 or the complement thereof. The nucleotide bases which are identical to those provided in SEQ ID NO:1 need not all be contiguous. However, in some embodiments, the nucleotide bases which are identical to SEQ ID NO:1 are contiguous. For example, a PMO of the present invention may comprise a sequence of 15 to 25 nucleotide bases of which at least 15, preferably at least 17, more preferably at least 20, most preferably at least 22, correspond to the nucleotide bases at the corresponding positions of SEQ ID NO:1 or the complement thereof such as

| | |
|---|---|
| TACGACTTACCGATGGTGTTATC | (SEQ ID NO: 3) |
| CCACAAACCCATGGTCATATC | (SEQ ID NO: 4) |
| ACAAACCCATGGTCA | (SEQ ID NO: 5) |
| CGTGCATGGGCACCAGTATTA | (SEQ ID NO: 6) |
| AACGTTTGGGTACCAGTATAT | (SEQ ID NO: 7) |

The method for the syntheses of the PMOs, the conjugation of P007, and the purification and analyses of P007-PMOs have all been described previously. See Moulton, H. M. et al. (2004) Bioconjug. Chem. 15:290-299, Summerton, J. (1999) Biochim. Biophys. Acta. 1489:141-158, and Summerton & Weller (1997) Antisense Nucleic Acid Drug Dev. 7:187-195, which are herein incorporated by reference.

To study the time dependent knock-down of targeted CD45, J774A.1 cells were either left untreated or treated with 8 μM of CD45 PMO or 8 μM of a scrambled control (SC PMO). At various time intervals, the cells were analyzed for CD45 expression by flow cytometry using methods known in the art.

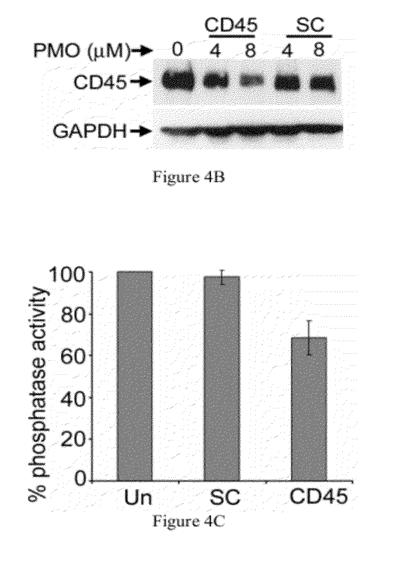

As shown in FIG. 4A, a maximum knock-down of CD45 was achieved within about 48 hours of 8 μM of CD45 PMO treatment (top panel), while 8 μM of PMO had no affect on CD45 expression levels (bottom panel). At 24, 48, 72 and 96 hours, the cells were stained and analyzed by flow cytometry methods known in the art. The cells (1×10⁶) were resuspended in Fc block (anti CD16/CD32 antibody diluted in RPMI containing 10% FBS) (BD Biosciences, San Diego, Calif.), incubated on ice for 30 minutes, centrifuged and stained with either isotype control antibody or FITC conjugated CD45 antibody (BD Biosciences, San Diego, Calif.). After incubation on ice for 60 minutes, the cells were washed and resuspended in 3.7% formaldehyde. FACS analysis was performed using a FACSCalibur flow cytometer (BD Biosciences, San Diego, Calif.) and methods known in the art. Data was analyzed using FlowJo software (Tree Star, Inc; Ashland Oreg.). Dose escalation studies showed that concentrations up to about 8 μM were not toxic to the cells (data not shown).

FIG. 4B shows a dose dependent reduction in CD45 protein levels in J774A.1 cells treated with CD45 PMO (CD45) as compared to untreated (0) or scrambled PMO (SC), when immunoblotted with CD45 antibody. The cells were harvested and lysed in buffer containing 50 mM Tris-HCl, pH 7.4, 150 mM NaCl, 2 mM EDTA, 1% Triton X-100, and protease inhibitor cocktail (Sigma, St. Louis, Mo.). The cell lysates were incubated for 30 minutes on ice and then centrifuged for 30 minutes at 14,000 rpm. Cell extracts (30 μg) were electrophoresed on SDS-PAGE and then subjected to Western blotting. A CD45 specific mouse monoclonal antibody (clone 69, BD Pharmingen, San Diego, Calif.) was used to detect the immunoreactive proteins that were visualized by chemiluminescence (ECL) methods known in the art. As a control for uniform protein loading, the bottom half of the blot was probed with GAPDH antibody (Ambion, Austin, Tex.).

Knock-down of CD45 expression levels by CD45 PMO was further confirmed by reduced phosphatase activity. Specifically, as shown in FIG. 4C, a concomitant reduction in CD45 phosphatase activity following immunoprecipitation of CD45 from protein lysates that were either untreated (Un) or treated with CD45 PMO (CD45) or SC PMO (SC) was observed. In these experiments, J774A.1 cells (about 1×10⁶) seeded in a 6 well plate were either left untreated or incubated with CD45 PMO (8 μM) or SC PMO (8 μM) for 72 hours. The cells were harvested and lysed in buffer containing 50 mM Tris-HCl, pH 7.4, 150 mM NaCl, 2 mM EDTA, 1% Triton X-100, and protease inhibitor cocktail (Sigma, St. Louis, Mo.). Equal concentration of total protein (200 μg) from untreated or treated cells were first pre-cleared with protein G Sepharose beads and then immunoprecipitated overnight with either a non-specific monoclonal antibody or a CD45 specific (clone 30-F11, BD Biosciences) antibody in the presence of protein G Sepharose beads. After washing, the beads were incubated with 100 μM of Difluorinated Methylumbelliferyl Phosphate (DiFMUP) substrate (Invitrogen, Carlsbad, Calif.) in 100 μl of assay buffer for 1 hour. Supernatant was transferred into 96-well plates and fluorescence intensity was measured at excitation 358 nm and emission 455 nm.

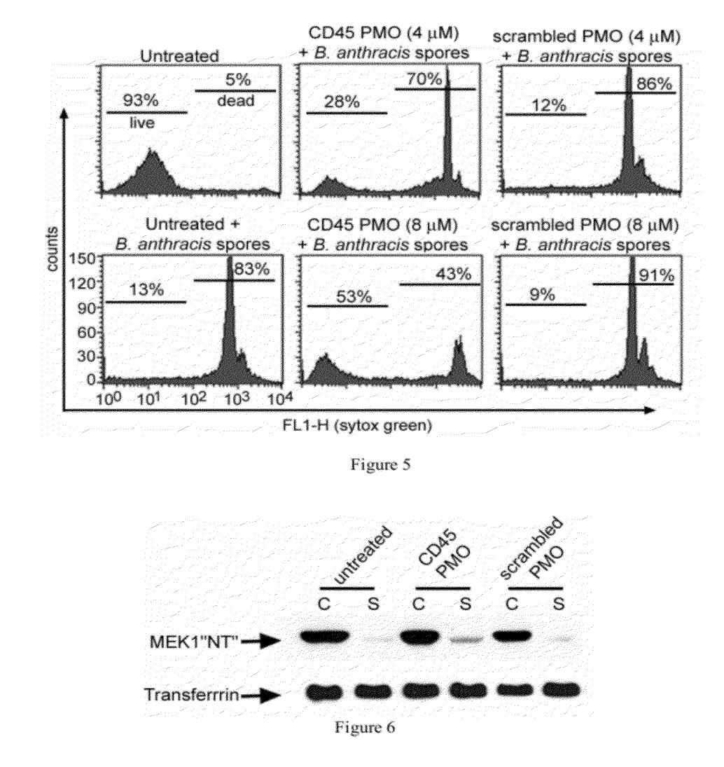

FIG. 5 shows that J774A.1 cells treated with CD45 PMO (CD45 PMO) showed increased viability against B. anthracis infection in a dose dependent manner when compared to the untreated cells or cells treated with SC PMO. In these experiments, J774A.1 cells (1×10⁵) were seeded in 24-well flat-bottom plates and incubated at 37° C./5% CO₂ in DMEM (Invitrogen, Carlsbad, Calif.) containing 10% FBS for 72 hours with SC PMO or CD45 PMO (4 or 8 μM). The cells were then harvested by manual scraping, placed into 1.5 ml tubes and infected with Sterne B. anthracis spores (MOI 5). After incubating for 4 hours at 37° C., bacterial growth was inhibited by the addition of antibiotics, penicillin (100 IU) and streptomycin (100 μg/ml). To determine cell viability, SYTOX green dye (1 μM, Invitrogen, Carlsbad, Calif.), which is impermeable to live cells, was added and incubated for 15 minutes at 37° C. The cells were centrifuged at 2000 rpm for 2 minutes and then washed two times with medium containing FBS (complete medium) and antibiotics. The cells were fixed with 1% formaldehyde for 15 minutes and then analyzed by flow cytometry known in the art. Thus, cells treated with CD45 PMO showed increased viability in a dose dependent manner. In contrast, J774A.1 cells that were not treated with PMO or treated with SC PMO were not protected from the direct cytotoxic effects of the anthrax lethal toxin (LT) (data not shown).

The different mitogen activated protein kinase kinase (MAPKK/MEK) isoforms have been shown to be the primary targets for anthrax LT proteolytic activity. See Duesbery, N. S. et al. (1998) Science 280:734-737, which is herein incorporated by reference. However, the observed protection against anthrax LT, as provided herein, does not correlate with MEK protection, as cells treated with CD45 PMO exhibited MEK cleavage pattern similar to control cells when infected with B. anthracis. Specifically, FIG. 6 is an immunoblot of cell lysates of J774A.1 cells untreated and treated with 8 μM CD45 PMO (CD45 PMO) or 8 μM SC PMO for 72 hours and subsequent infection with Sterne B. anthracis spores for 4 hours which shows that MEK cleavage was not prevented in cells treated with PMOs. In these experiments, J774A.1 cells were harvested and lysed in buffer containing 50 mM Tris-HCl, pH 7.4, 150 mM NaCl, 2 mM EDTA, 1% Triton X-100, and protease inhibitor cocktail (Sigma, St. Louis, Mo.). The cell lysates were incubated for 30 minutes on ice and then centrifuged for 30 minutes at 14,000 rpm. The cell extracts (30 μg) were electrophoresed on SDS-PAGE and then subjected to Western blotting. The cell lysates were immunoblotted with MEK1″NT″ antibody (Upstate, Lake Placid, N.Y.). In the immunoblot of FIG. 6, lanes C are lysates from uninfected cells and lanes S are lysates from infected cells and the top half of the immunoblot was incubated with the anti-transferrin receptor antibody (Invitrogen, Carlsbad, Calif.) as a control for uniform protein loading. These findings indicate that the induction of macrophage cell death during B. anthracis infection may involve a tyrosine phosphorylation dependent pathway that is independent of the MEK pathway.

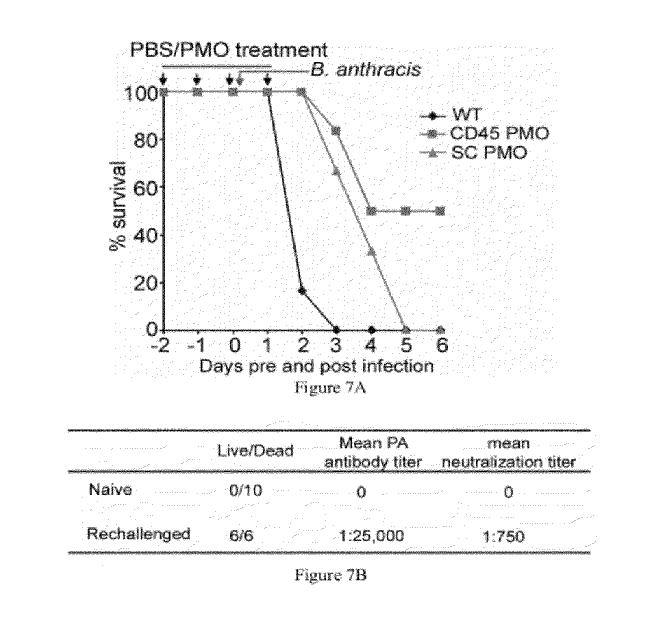

To further confirm the importance of CD45 in anthrax pathogenesis, BALB/c mice (6-8 weeks old, n=6) were pretreated via subcutaneous (s.c.) route with phosphate buffered saline (PBS) or CD45 PMO or SC PMO for 2 days (day −2, −1). On day 3 (day 0), the mice were challenged via intraperitoneal (i.p.) route with 3LD₅₀ Ames B. anthracis spores and treated s.c. with additional PBS or CD45 PMO or SC PMO. A final treatment with the PMOs or PBS was given one day post challenge (day 1). Non-tagged PMOs were used for these studies and were injected at a dose of 100 mg/kg/day. The mice were monitored for 1 month post challenge. Survival was greatly increased in the animals treated with CD45 PMO compared to those treated with PBS or SC PMO. See FIG. 7A.

The mice treated with CD45 PMO and that survived infection developed protective antigen specific and lethal toxin neutralizing antibody titers and were completely protected when re-challenged with Ames B. anthracis spores, as shown in FIG. 7B. Neutralizing assays and antibody detection assays were carried out using methods known in the art. See Little, S. F. et al. (1996) Microbiology 142 (Pt 3):707-715, which is herein incorporated by reference.

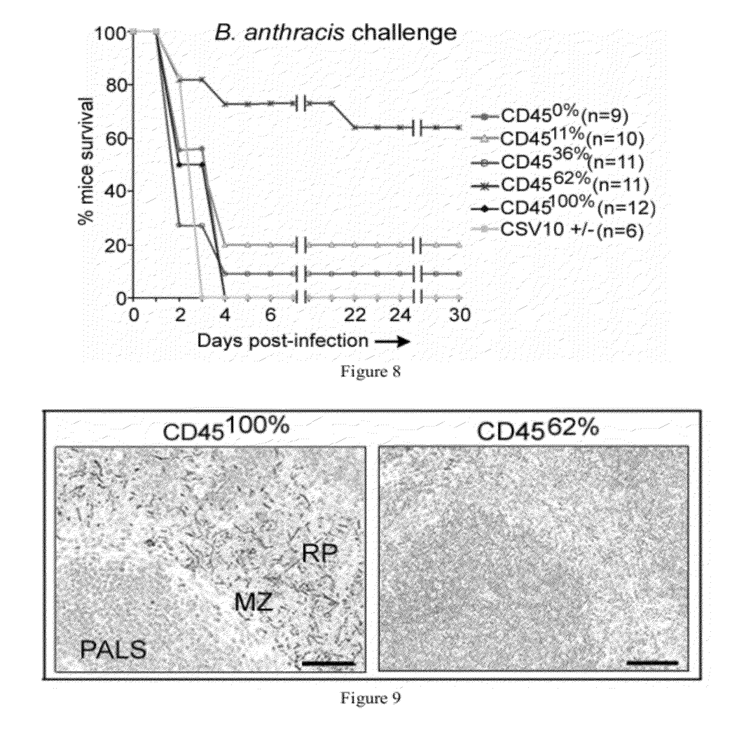

FIG. 8 shows that mice with reduced CD45 expression (62% CD45 expression) showed an increased survival rate of 65%, but wild type (CD45^{100%}) mice, CD45 knockout mice (CD45^{0%}), and transgenic mice with 62% CD45 expression but no phosphatase activity (CSV10 +/−) did not survive B. anthracis infection.

Mice that survived 48 hours (FIG. 9, right panel) after infection with B. anthracis or 30 days post-challenge (data not shown) showed no evidence of bacilli in the spleens (FIG. 9, right panel), lymph nodes and lungs (data not shown), as observed by immunohistochemical staining with anti-capsule antibody. In contrast, spleens from moribund CD45^{100%} mice showed a heavy bacterial burden in the red pulp (RP) areas with fewer bacilli appearing in cells associated with the marginal zone (MZ) (FIG. 9, left panel) and no visible bacilli in periarteriolar lymphoid sheaths (PALS). In these experiments, the presence of bacilli in infected tissues was detected using the EnVision systems (Dako, Carpinteria, Calif.). Briefly, the tissue sections were deparaffinized, blocked in methanol/H₂O₂ solution for 30 minutes at room temperature, rinsed with water, pretreated with Tris-EDTA, pH 9.0 at 97° C. for 30 minutes and then blocked with the mouse IgG blocking buffer (Vector Labs, Burlingame, Calif., 1:20). The tissues were then incubated with mouse anti-capsule antibody or mouse IgG as negative control serum for 30 minutes at room temperature. After rinsing three times with PBS, peroxidase labeled polymer conjugated to goat anti-mouse immunoglobulins was added and incubated for 30 minutes. After rinsing with PBS, substrate-chromogen solution (Dako, Carpinteria, Calif.) was added, incubated for 5 minutes, rinsed with PBS, stained with hematoxylin (Dako, Carpinteria, Calif.), dehydrated and tissues were mounted with Permount (Fisher Scientific, Fair Lawn, N.J.).

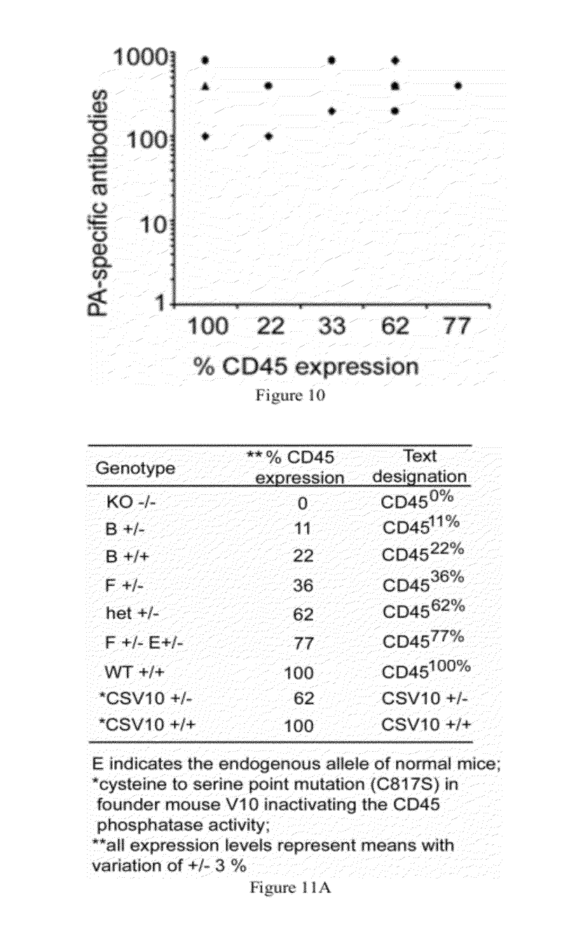

None of the survivor mice (CD45^{11-62%} mice) challenged with B. anthracis developed protective antigen (PA)-specific antibody titers, thereby suggesting an early clearance of the bacteria (data not shown). However, mice with reduced CD45 expression were capable of mounting an immune response to B. anthracis antigens as demonstrated by the generation of PA-specific antibodies after vaccination with anthrax vaccine adsorbed (AVA) as shown in FIG. 10. The PA specific antibody responses were measured by ELISA using methods known in the art. See Little, S. F. et al. (1996) Microbiology 142 (Pt 3):707-715, which is herein incorporated by reference. A survival rate of 50% (4/8) was observed when mice (CD45^{11-62%} mice) that survived the first challenge were re-challenged with Ames B. anthracis spores.

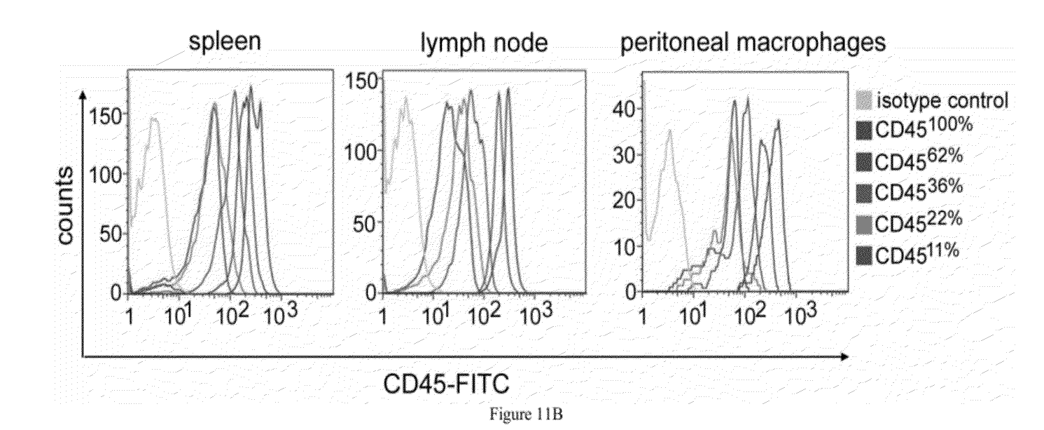

Mice engineered to express different levels of CD45 and used in this study are listed in FIG. 11A. FIG. 11B depicts the CD45 expression levels in transgenic and heterozygous mice. Peritoneal macrophages and cells isolated from spleen and lymph nodes were stained with FITC-conjugated CD45 antibody and analyzed by flow cytometry in accordance with Example 3 below.

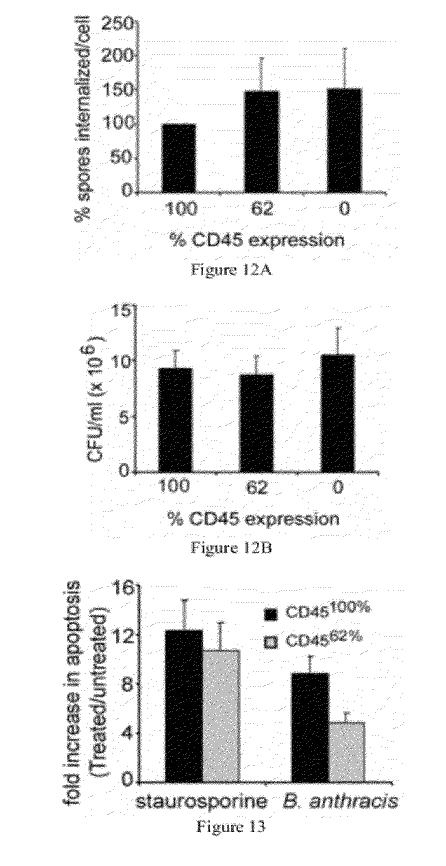

To investigate if reduced CD45 levels disrupted the biological functions of immune cells, the phagocytic and killing properties of peritoneal macrophages were measured. Macrophages harvested from the CD45^{62%} or CD45^{0%} mice possessed the ability to internalize the spores and kill the bacteria as efficiently as those from CD45^{100%} mice. Specifically, as provided in FIG. 12A, reduced CD45 expression does not affect the functional properties of immune cells in mice. In these experiments, thioglycolate elicited peritoneal macrophages from CD45^{100%} mice, CD45^{62%} mice and CD45^{0%} mice were infected with 5 MOI of GFP-Sterne spores and plate centrifuged to synchronize the infection. After 30 minutes, non-permeabilized cells were incubated with a mixture of antibodies specific for B. anthracis spore exosporium (to label extracellular spores) and bacillus polysaccharide (to label extracellular vegetative bacilli) (kindly provided by T. Abshire and J. Ezzel, USAMRIID), followed by a secondary incubation with antibody conjugated to Alexa-594 nm fluorophore (Invitrogen, Carlsbad, Calif.). Thus, only spores adhered to the outside surface of the macrophages are labeled. After fixation with formaldehyde, the cells were stained with Hoechst dye (Invitrogen, Carlsbad, Calif.) and images from nine sites/well were collected and analyzed using the Discovery-1 high-content screening system (Molecular Devices, Downington, Pa.). Images were analyzed using the cell health module of MetaXpress imaging analysis software (Molecular Devices, Downington, Pa.). Total cell count was based on the number of Hoechst-stained cell nuclei, while co-localization with red (anti-spore and anti-bacterial antibody) and GFP-Sterne spores was scored as spores being on the outside of the cell and co-localization with GFP-Sterne spores are ingested spores. The data represents averages from three independent experiments and indicates the percentage of spores internalized/cell.

As provided in FIG. 12B, reduced CD45 expression does not affect the killing properties of the macrophages. In these experiments, thioglycolyate elicited peritoneal macrophages from CD45^{100%} mice, CD45^{62%} mice and CD45^{0%} mice were infected with Sterne B. anthracis spores (MOI 5). After 30 minutes, cell pellets collected by centrifugation were lysed in sterile water, serially diluted and aliquots were plated onto solid LB agar medium plates, which were then incubated at 37° C. for 16 hours. Colony forming units (CFU) were counted and data are represented as CFU/ml. The data represents averages from three independent experiments±standard deviation (s.d.).

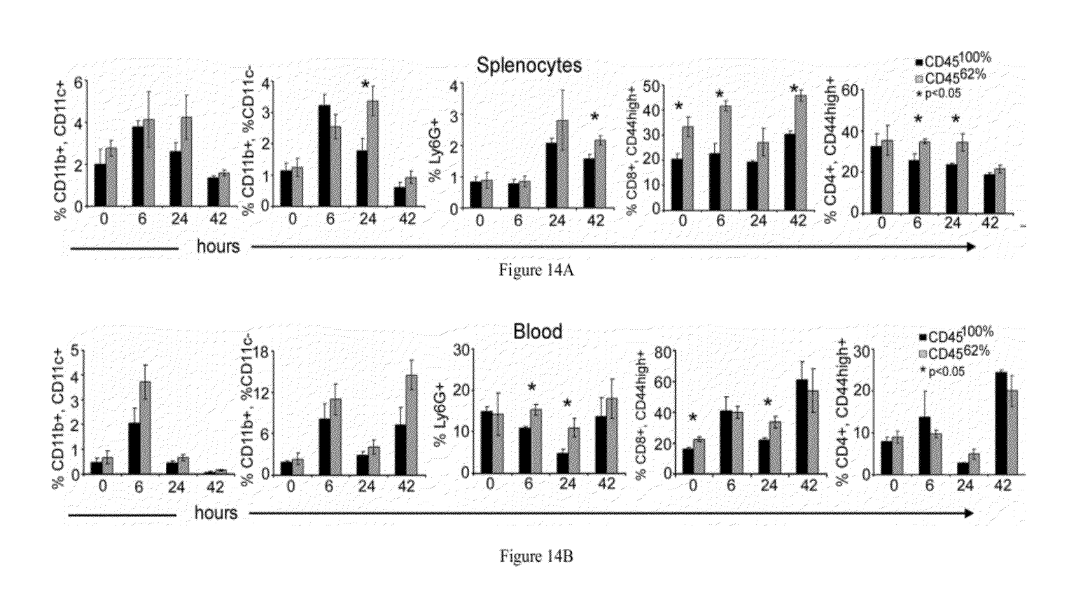

Interestingly, as shown in FIG. 13, CD45^{62%} macrophages infected with B. anthracis exhibited reduced apoptosis compared to their CD45^{100%} controls as measured by levels of activated caspase 3/7, thus suggesting that reduced CD45 levels may regulate, reduce, or inhibit apoptosis in these cells. In these experiments, thioglycolate (BD Biosciences, San Jose, Calif.) elicited peritoneal macrophages from CD45^{100%} or CD45^{62%} mice were seeded in DMEM (Invitrogen, Carlsbad, Calif.) containing 10% FBS at 10⁵ cells/100 μl per well in 96-well format. The cells were either left untreated, infected with B. anthracis spores (10 MOI) or treated with staurosporine (2 μM, EMD Biosciences, San Diego, Calif.) in triplicate wells for 6 hours and then examined for apoptosis using an Apo-One homogenous Caspase-3/7 kit (Promega, Madison, Wis.) using methods known in the art. The fold increase in apoptosis represents the ratio of treated to untreated cells.

To understand the role of CD45 in B. anthracis infected mice, a time-course study to monitor functional and cellular changes was conducted. Measurement of twenty host cytokine and chemokine responses in the plasma of CD45^{100%} and CD45^{62%} mice induction of cytokines (IL-10, IL-12, IL-13) in both CD45^{100%} and CD45^{62%} mice. However there were no clear differences for these cytokines between the two groups of mice (data not shown).

As shown in FIG. 14A, cell profiling studies of splenic cells indicated a significant increase in the percentage of CD11b⁺ macrophages (24 hours), Ly6G⁺ granulocytes (42 hours), CD8⁺ CD44^{high+} T cells (0, 6 and 42 hours) and CD4⁺ CD44^{high+} T cells (6 and 24 hours) in CD45^{62%} mice. In these experiments, splenocytes were isolated from mice euthanized at time 0, 6, 24 and 42 hours post B. anthracis challenge. Antibodies used for FACS analysis were purchased from BD Pharmingen (San Diego, Calif.), unless otherwise noted. The cells (1×10⁶) were resuspended in Fc block (anti-CD16/CD32 antibody diluted in RPMI containing 10% FBS), incubated on ice for 30 minutes, centrifuged and stained with appropriate combinations of labeled antibodies. After incubation on ice for 60 minutes, the cells were washed and resuspended in 3.7% formaldehyde. FACS analysis was performed using a FACSCalibur flow cytometer (BD Biosciences, San Diego, Calif.). Data was analyzed using FlowJo software (Tree Star, Inc; Ashland, Oreg.).

In blood samples an increased percentage of Ly6G⁺ granulocytes (6 and 24 hours) and CD8⁺ CD44^{high+} T cells (0, 24 hours) was also observed in the CD45^{62%} mice as shown in FIG. 14B. In these experiments, blood cells collected from mice euthanized at time 0, 6, 24 and 42 hours post B. anthracis infection were stained for cell surface and activation markers and analyzed by flow cytometry using methods known in the art. Antibodies used for FACS analysis were purchased from BD Pharmingen (San Diego, Calif.), unless otherwise noted. Whole blood was lysed in equal volume ACK Lysis Buffer (Cambrex, Walkersville, Md.) for 2 minutes and washed 2 times with RPMI containing 10% FBS. Cells (1×10⁶) were resuspended in Fc block (anti-CD16/CD32 antibody diluted in RPMI containing 10% FBS), incubated on ice for 30 minutes, centrifuged and stained with appropriate combinations of labeled antibodies. After incubation on ice for 60 minutes, cells were washed in complete medium and resuspended in 3.7% formaldehyde. FACS analysis was performed using a FACSCalibur flow cytometer (BD Biosciences, San Diego, Calif.). Data was analyzed using FlowJo software (Tree Star, Inc; Ashland, Oreg.).

Thus, these time course studies suggest that reduced expression levels of CD45 alter the immune responses in the CD45^{62%} mice infected with B. anthracis, through increased numbers of innate immune cells (macrophages, granulocytes) and activated T cells. This indicates that innate immune cells may play a central role in the clearance of B. anthracis infection. Overall, these in vivo, functional and profiling results indicate that expression levels of CD45 may be reduced in order to prevent, modulate, inhibit, treat, or reduce pathogenesis and morbidity associated with an infection with a biological agent, such as B. anthracis.

### Viral Infection

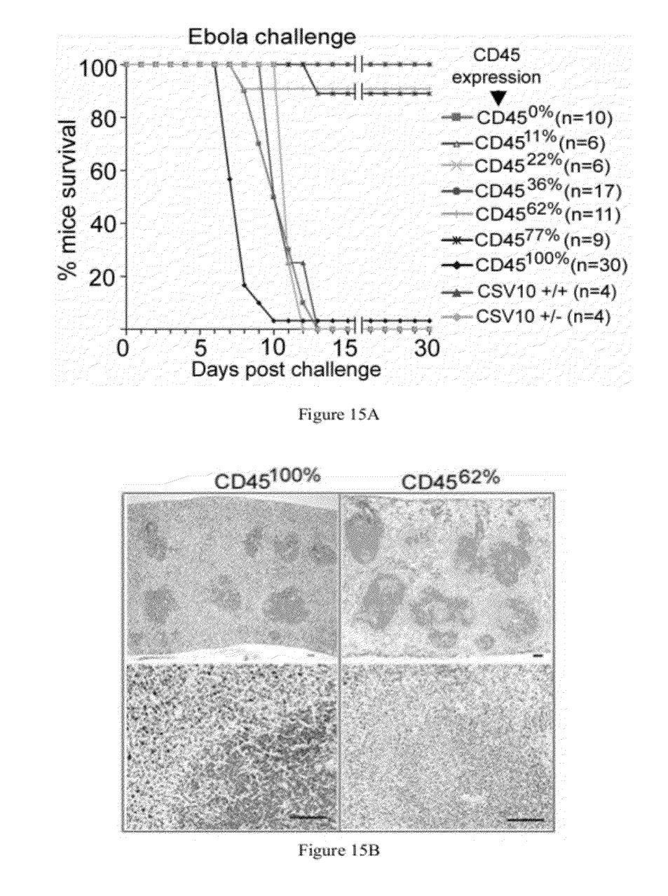

To determine if alterations in expression levels of CD45 could lead to protection against infections by a virus belonging to Filoviridae, mice with reduced CD45 expression levels were infected with EBOV. In these experiments, mice were challenged via intraperitoneal (i.p.) route with 1000 pfu of mouse adapted Ebola Zaire virus (EBOV). See Bray, M. et al. (1998) J. Infect. Dis. 178:651-661, which is herein incorporated by reference. Mice having CD45 expression levels ranging from about 11% to about 77% (CD45^{11%}, CD45^{22%}, CD45^{36%}, CD45^{62%}, CD45^{77%}) had about a 90% to about a 100% survival rate, whereas the CD45^{100%}, CD45^{0%} and CSV10 mice did not survive EBOV challenge as shown in FIG. 15A. A delay, however, in the mean time-to-death was observed in CD45^{0%} mice and CSV10 mice.

As shown in FIG. 15B, the CD45^{62%} mice (right panel) and other mice with reduced CD45 expression (data not shown) that survived 30 days after challenge with EBOV cleared the virus in various organs. In contrast, as shown in FIG. 15B (left panel), the spleens of moribund CD45^{100%} mice were heavily infected with EBOV (day 7). In these experiments, spleens from CD45^{100%} mice (left panel) and CD45^{62%} mice (right panel) were stained with anti-EBOV antibody. The tissue sections were deparaffinized, blocked in methanol/H₂O₂ solution for 30 minutes at room temperature, rinsed with water and pretreated with freshly diluted Proteinase K solution (20 μg/ml) (Sigma-Aldrich, St. Louis, Mo.) for 15 minutes at room temperature. After blocking with the CAS block containing 5% goat serum (Vector, Burlingame, Calif.), the tissue sections were incubated with rabbit anti-Ebola antibody for 60 minutes at room temperature. After rinsing three times with PBS, peroxidase labeled polymer conjugated to goat anti-rabbit immunoglobulins (DAKO, Carpinteria, Calif.) was added and then incubated for 30 minutes. After rinsing with PBS, substrate-chromogen solution (DAKO, Carpinteria, Calif.) was added, incubated for 5 minutes, rinsed with PBS, stained with hematoxylin (DAKO, Carpinteria, Calif.)), dehydrated and tissues were mounted with Permount (Fisher Scientific, Fair Lawn, N.J.).

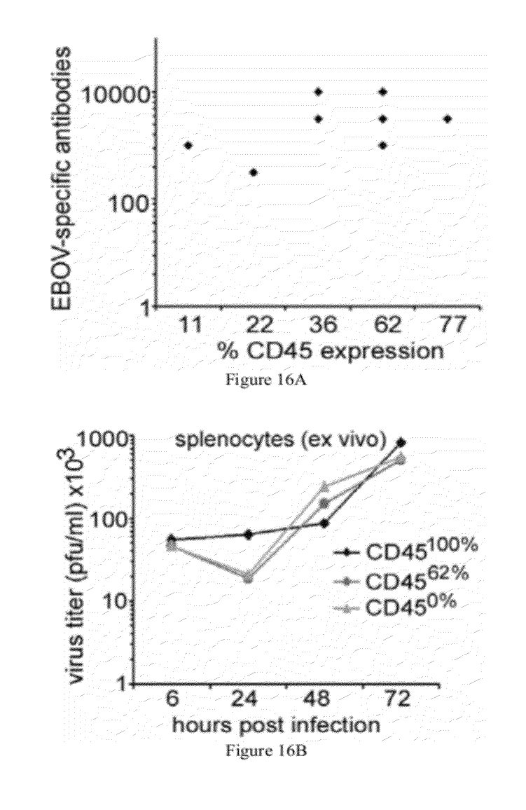

FIG. 16A shows CD45^{100%}, CD45^{62%}, CD45^{36%}, CD45^{22%}, and CD45^{11%} mice surviving EBOV challenge generated serum EBOV-specific antibody responses. Briefly, blood samples were obtained from the retro-orbital sinus under anesthesia, lateral tail vein, or by cardiac puncture under anesthesia, and serum was collected and stored at −70° C. Levels of EBOV-specific antibodies were determined using methods known in the art. See Hevey et al. (1997) Virology 239:206-216, which is herein incorporated by reference. Briefly, the wells were coated with sucrose-purified inactivated EBOV. Serial 3-fold dilutions of individual mice serum were tested and detected using an HRP-conjugated Ab to measure total (IgA, IgG, IgM; Sigma-Aldrich) or the individual isotype (IgA or IgM) or IgG subclass antibody levels (Southern Biotechnology Associates), and developed using tetramethylbenzidine substrate. Antibody titers were defined as the reciprocal of the highest dilution showing a net OD ≧0.2. Mice that survived EBOV challenge also developed CD8⁺ T cell responses to defined EBOV GP, NP, and VP40 epitopes (data not shown). Upon re-challenge with EBOV, a 100% survival rate (20/20) was observed for mice expressing a range of CD45 levels (11% to 77%). As shown in FIG. 16B, ex vivo EBOV infection of splenocytes obtained from CD45^{100%}, CD45^{62%} and CD45^{0%} mice showed similar viral titers suggesting that changes in CD45 cell surface expression levels had no effect on viral replication. See FIG. 16B. In these experiments, splenocytes harvested from CD45^{100%} mice, CD45^{62%} mice and CD45^{0%} mice were infected (MOI=1) ex vivo with mouse adapted EBOV. At times 6, 24, 48 and 72 hours, supernatant was harvested and viral titers were determined by plaque assays known in the art. See Moe, J. B. et al. (1981) J. Clin. Microbiol. 13:791-793, which is herein incorporated by reference.

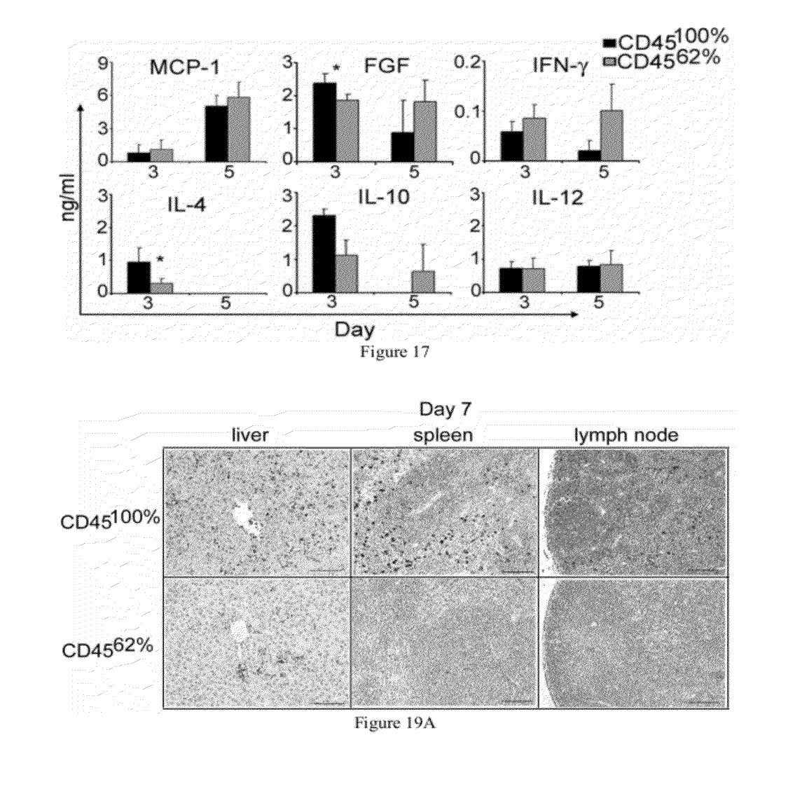

To understand the role of CD45 in mice infected with EBOV, a time-course study to monitor functional and cellular changes at both the transcript and protein level was conducted. As shown in FIG. 17, cytokines and chemokines, MCP-1, FGF, IFN-γ, IL-4, IL-10 and IL-12, were induced in the middle stages of the infection (day 3 and/or day 5) in both the CD45^{62%} and CD45^{100%} mice. In these experiments, the plasma of CD45^{100%} and CD45^{62%} mice euthanized at days 0, 1, 3 and 5 post infection were analyzed with cytokine 20-plex luminex kit (Invitrogen, Carlsbad, Calif.) according to the manufacturer instructions and methods known in the art. All analyses were performed with a Bio-Plex workstation and the accompanying software (Bio-Rad, Hercules, Calif.). Interestingly, 3 days after infection there was a significant difference in the IL-4 levels between the CD45^{100%} mice and the CD45^{62%} mice, and the IL-4 were abolished by day 5 in both groups of mice. By day 3 post challenge, the CD45^{100%} mice had increased IL-10 levels compared to CD45^{62%} mice. IL-10 levels were completely abrogated by day 5 post-infection in the CD45^{100%} mice. These data suggest the initiation of active immunity in the CD45^{62%} mice regulates EBOV infection in vivo.

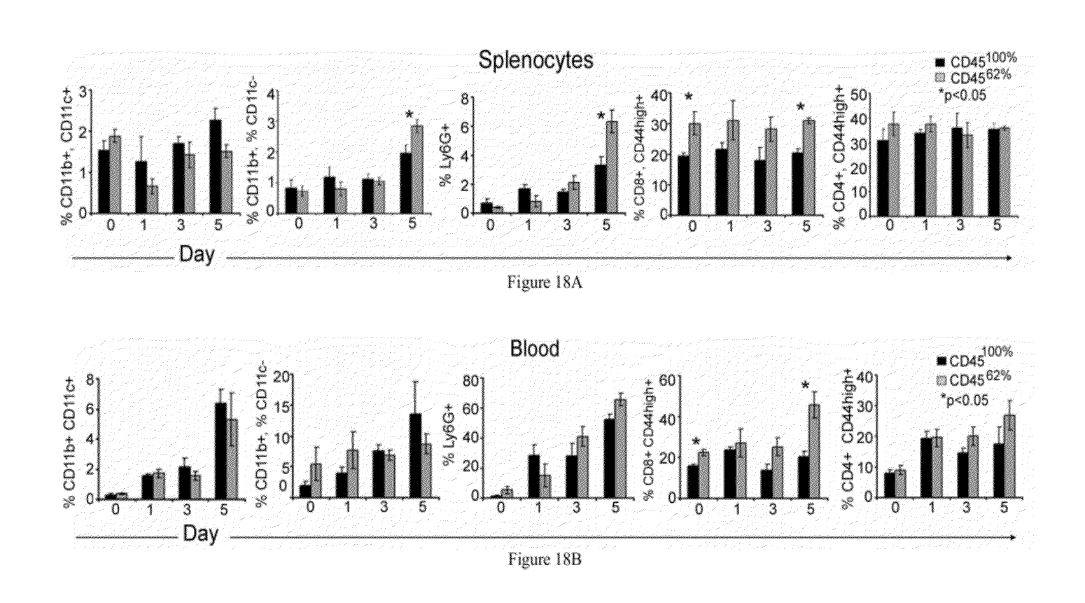

FIG. 18A shows that splenocytes isolated from mice post EBOV infection exhibited a significant increase in the percentage of CD11b⁺ macrophages, Ly6G⁺ granulocytes at day 5 post EBOV challenge and CD8⁺ CD44^{high+} T cells at days 0 and 5 post challenge in the CD45^{62%} mice versus the CD45^{100%} mice. In these experiments, splenocytes were isolated from mice euthanized at days 0, 1, 3 and 5 post-EBOV infection and then stained for cell surface and activation markers and analyzed by flow cytometry using methods known in the art. Antibodies used for FACS analysis were purchased from BD Pharmingen (San Diego, Calif.), unless otherwise noted. The cells (1×10⁶) were resuspended in Fc block (anti-CD16/CD32 antibody diluted in RPMI containing 10% FBS), incubated on ice for 30 minutes, centrifuged and stained with appropriate combinations of labeled antibodies. After incubation on ice for 60 minutes, the cells were washed and resuspended in 3.7% formaldehyde. FACS analysis was performed using a FACSCalibur flow cytometer (BD Biosciences, San Diego, Calif.). Data was analyzed using FlowJo software (Tree Star, Inc; Ashland, Oreg.).

FIG. 18B shows that in the blood samples, the percentage of activated CD8⁺ CD44^{high+} T cells was increased (day 0-5 post challenge) in the CD45^{62%} mice compared to the CD45^{100%} mice. In these experiments, blood was collected from mice euthanized at days 0, 1, 3 and 5 post EBOV infection and then stained for cell surface and activation markers and analyzed by flow cytometry using methods known in the art. Antibodies used for FACS analysis were purchased from BD Pharmingen (San Diego, Calif.), unless otherwise noted. Whole blood was lysed in equal volume ACK Lysis Buffer (Cambrex, Walkersville, Md.) for 2 minutes and washed 2 times with RPMI containing 10% FBS. The cells (1×10⁶) were resuspended in Fc block (anti-CD16/CD32 antibody diluted in RPMI containing 10% FBS), incubated on ice for 30 minutes, centrifuged and stained with appropriate combinations of labeled antibodies. After incubation on ice for 60 minutes, the cells were washed in complete medium and resuspended in 3.7% formaldehyde. FACS analysis was performed using a FACSCalibur flow cytometer (BD Biosciences, San Diego, Calif.). Data was analyzed using FlowJo software (Tree Star, Inc; Ashland, Oreg.).

The results of these time course studies suggest that infection by EBOV alters the immune responses in the CD45^{62%} mice, thereby resulting in an increased percentage of innate immune cells (macrophages and granulocytes) and activated T cells. Collectively, the data suggest that CD45 regulates active homeostasis of immune cells during infection with a filovirus.

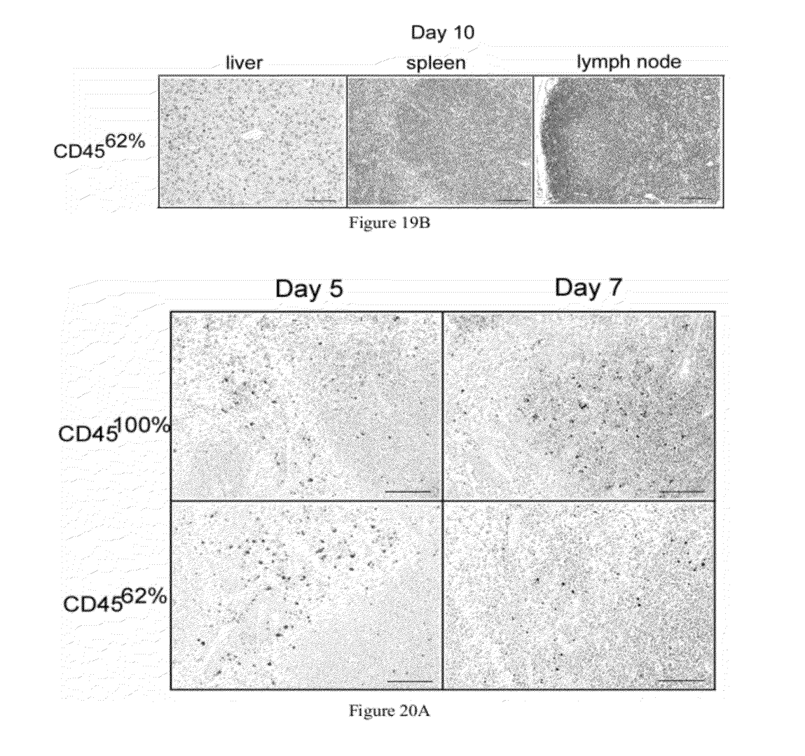

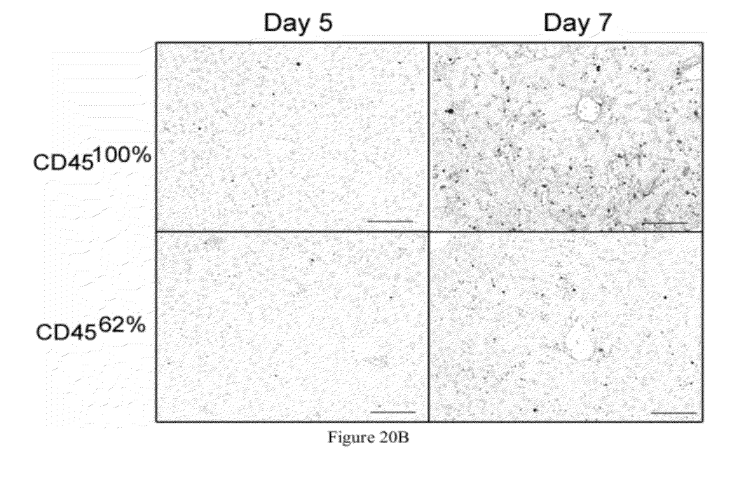

To monitor the viral load in the CD45^{100%} mice and the CD45^{62%} mice at different stages of EBOV infection, tissues from the time course study were stained for viral burden. FIG. 19 shows immunohistochemical stained liver, spleen and lymph node tissues which showed EBOV antigen-positive cells in CD45^{100%} mice at day 7 post challenge. In contrast the CD45^{62%} mice showed reduced viral antigen at day 7 post-challenge. No EBOV antigen was detected by day 10 post challenge in the CD45^{62%} mice. FIG. 20A, shows apoptosis in spleen of CD45^{100%} and CD45^{62%} mice as observed by TUNEL technique. TUNEL staining of spleen from CD45^{100%} and CD45^{62%} mice on day 5 post EBOV challenge revealed apoptotic cells in areas where extramedullary hematopoiesis (EMH) normally occurs. Significant cell depletion was observed on day 5 post infection suggesting prior loss of cells. By day 7 reduction in number of apoptotic cells was observed in CD45^{62%} mice compared to CD45^{100%} mice. FIG. 20B shows TUNEL staining of liver showed increased apoptosis by day 7 in the CD45^{100%} mice. In contrast there was greatly reduced apoptosis in CD45^{62%} mice by day 7 post challenge.

In these experiments, to detect the presence of EBOV in infected organs, the tissue sections were deparaffinized, blocked in methanol/H₂O₂ solution for 30 minutes at room temperature, rinsed with water and pretreated with freshly diluted 20 μg/ml Proteinase K solution (DAKO, Carpinteria, Calif.) for 15 minutes at room temperature. After blocking with the CAS block containing 5% goat serum (Vector, Burlingame, Calif.), the sections were incubated with rabbit anti-Ebola antibody for 60 minutes at room temperature. After rinsing three times with PBS, peroxidase labeled polymer conjugated to goat anti-rabbit immunoglobulins (DAKO, Carpinteria, Calif.) was added and incubated for 30 minutes. After rinsing with PBS, substrate-chromogen solution (DAKO, Carpinteria, Calif.) was added, incubated for 5 minutes, rinsed with PBS, stained with hematoxylin (DAKO, Carpinteria, Calif.), dehydrated and tissues were mounted with Permount (Fisher Scientific, Fair Lawn, N.J.).

Apoptosis in tissues was detected by terminal uridine deoxynucleotidyl transferase dUTP nick end labeling (TUNEL) using the APOPTAG® Plus Peroxidase In Situ Apoptosis Kit (Chemicon, Temecula, Calif.). Processing of the tissue samples was carried out as described above, with the following changes. After proteinase K treatment and washing, tissues were incubated with terminal deoxynucleotidyl transferase (TdT) (Chemicon, Temecula, Calif.) for 60 minutes at 37° C. The reaction was stopped by adding stop/wash buffer (Chemicon, Temecula, Calif.) and incubating for 10 minutes at room temperature. After washing three times with PBS, the tissues were incubated with anti-digoxigenin-peroxidase (Chemicon, Temecula, Calif.) for 30 minutes at room temperature. Color development was visualized after incubation with the DAB substrate (Chemicon, Temecula, Calif.). Slides were counterstained in methyl green.

FIG. 20 (left panel) shows immunohistochemical stains of mesenteric lymph node (mLN) tissue having EBOV in discrete foci within the cortices in both the CD45^{100%} mice and the CD45^{62%} mice on day 5 post EBOV challenge (left panels). There was no difference in either the number of foci or in the amount of viral antigen for the CD45^{100%} mice and the CD45^{62%} mice by day 5 post EBOV challenge. Staining for apoptosis in the mLN (FIG. 20, right panel), showed TUNEL positive cells in the cortices on days 5 post EBOV infection in both the CD45^{100%} and CD45^{62%} (FIG. 20, right panel).

In these experiments, to detect the presence of EBOV in organs, the tissue sections were deparaffinized, blocked in methanol/H₂O₂ solution for 30 minutes at room temperature, rinsed with water and pretreated with freshly diluted Proteinase K solution (20 μg/ml) (DAKO, Carpinteria, Calif.) for 15 minutes at room temperature. After blocking with the CAS block containing 5% goat serum (Vector, Burlingame, Calif.), the sections were incubated with rabbit anti-Ebola antibody for 60 minutes at room temperature. After rinsing three times with PBS, peroxidase labeled polymer conjugated to goat anti-rabbit immunoglobulins (DAKO, Carpinteria, Calif.) was added and incubated for 30 minutes. After rinsing with PBS, substrate-chromogen solution (DAKO, Carpinteria, Calif.) was added, incubated for 5 minutes, rinsed with PBS, stained with hematoxylin (DAKO, Carpinteria, Calif.), dehydrated and tissues were mounted with Permount (Fisher Scientific, Fair Lawn, N.J.).

Apoptosis in tissues was detected by TUNEL using the APOPTAG® Plus Peroxidase In Situ Apoptosis Kit (Chemicon, Temecula, Calif.) as described above, but with the following changes. After proteinase K treatment and washing, the tissues were incubated with terminal deoxynucleotidyl transferase (TdT) (Chemicon, Temecula, Calif.) for 60 minutes at 37° C. The reaction was stopped by adding stop/wash buffer (Chemicon, Temecula, Calif.) and then incubating for 10 minutes at room temperature. After washing three times with PBS, the tissues were incubated with anti-digoxigenin-peroxidase (Chemicon, Temecula, Calif.) for 30 minutes at room temperature. Color development was visualized after incubation with the DAB substrate (Chemicon, Temecula, Calif.). Slides were counterstained in methyl green (PolyScientific, Bay Shore, N.Y.).

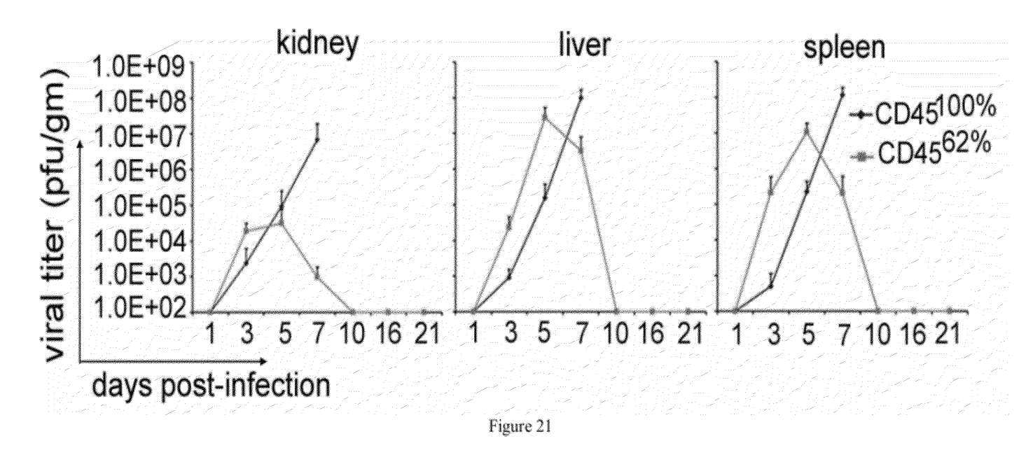

Viral titers in spleen, liver and kidney tissues were measured using plaque forming assays known in the art. See Moe, J. B. et al. (1981) J. Clin. Microbiol. 13:791-793, which is herein incorporated by reference. These studies revealed that the viral titer in the CD45^{62%} mice begins to drop after day 5 post-infection and nearly complete to complete clearance of the virus was observed by day 10 post-infection. See FIG. 21.

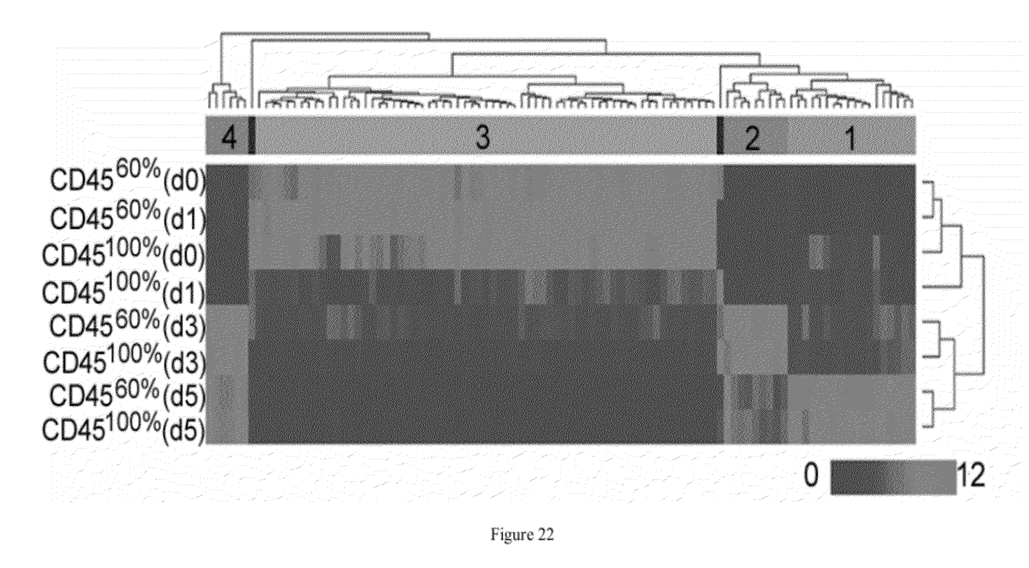

To investigate genes modulated by EBOV, an Affymetrix gene chip array was used to compare global gene expression changes in the CD45^{100%} mice and the CD45^{62%} mice. In these experiments, splenocytes isolated from spleen of wild type (100%) and heterozygous (62%) mice at day 0, 1, 3 and 5 post-Ebola Zaire infection were resuspended in 2 ml TRIzol solution (Invitrogen, Carlsbad, Calif.). The samples were stored at −70° C. until the RNA was purified. Total cellular RNA was isolated as per the manufacturer's specifications. The quality and concentration of the RNA were determined by measuring the absorbance at 260 and 280 nm. RNA integrity was confirmed by an Ailent 2100 Bioanalyzer (Agilent technologies, Palo Alto, Calif., USA). The mouse genome 430 2.0 array (Affymetrix, Santa Clara, Calif.), which comprises over 39,000 genes in a single array, was used. Affymetrix CEL files were preprocessed using GCRMA in R/Bioconductor. See Wu et al. (2003) J. Amer. Stat. Assoc. 99:909, which is herein incorporated by reference. Median log2 expression values within each sample type/time point with coefficient of variation (CV) >0.4 were chosen for agglomerative hierarchical clustering. Genes on the X or Y chromosome were removed and the probeset with the largest CV was chosen for genes with >1 probeset. Cluster stability was assessed with clusterStab to suggest cluster boundaries. As color version of FIG. 22 may be found on the World Wide Web at 69.89.17.19/˜datacons/sbavari/figures.pdf, which is herein incorporated by reference, and shows genes and samples arranged by dendrograms created with agglomerative nesting and assigned to 4 main clusters (pink, green, blue, orange bars). Each gene cluster was associated with cellular immune processes, signaling, cell-cycle, complement coagulation cascade, biosynthesis/metabolism, ubiquitous genes involved in several cascades and genes of unknown origin. Two genes are not members of any cluster (black bars).

FIG. 22 shows that gene modulation patterns of variable genes have been grouped into four main clusters, with each gene cluster associated with cellular immune responses, signaling, cell-cycle, complement coagulation cascade, biosynthesis/metabolism, ubiquitous genes involved in several cascades and genes of unknown origin. Interestingly, gene expression in cluster 3 was significantly down-modulated by day 1 in the CD45^{100%} mice. In contrast, at day 1 post EBOV infection, the CD45^{62%} maintained normal gene expression similar to day 0. Interestingly, the results as shown in FIG. 22, show that gene expression in cluster 3 a delayed host response to EBOV in the CD45^{62%} mice. Specifically, the gene expression profile for day 1 post-infected CD45^{62%} mice was similar to the non-infected (day 0) mice. The differences in the gene expression between the CD45^{100%} and CD45^{62%} were still apparent at day 3 post-infection but by day 5 the gene expression patterns were very similar.

The following examples are intended to illustrate but not to limit the invention.

### Example 1

### Transgenic Mice

Transgenic mice expressing reduced levels of CD45 were produced using a CD45 minigene construct containing cDNA for exons 1b-3, the genomic sequence from exon 3 to exon 9 which includes the variably spliced exons and surrounding introns, and cDNA from exon 9 through the polyadenylation signal region in exon 33 as described previously. See Virts, E. L. et al. (2003) Blood 101:849-855 and Virts & Raschke (2001) J. Biol. Chem. 276:19913-19920, which are herein incorporated by reference. Three founder transgenic mice were obtained, B, F and H and each were bred onto the exon-9 disrupted CD45 knockout strain obtained from Jackson Labs. See Byth, K. F. et al. (1996) J. Exp. Med. 183:1707-1718, which is herein incorporated by reference. The properties of the F strain have been reported by Virts et al. See Virts, E. L. et al. (2003) Blood 101:849-855, which is herein incorporated by reference. Transgenic mice containing a point mutation (C817S) in the membrane proximal phosphatase domain of the CD45 minigene were also produced. This mutation has been shown to abolish CD45 PTPase activity in vitro and has been confirmed in ex vivo studies. See Desai, D. M. et al. (1994) EMBO J. 13:4002-4010, which is herein incorporated by reference.

The CSV10 transgenic mice were generated using the C817S mutant minigene and the transgene locus was bred onto the CD45 knockout background using methods known in the art.

### Example 2

### Phosphatase Activity Assays

Protein phosphatases were purchased from Upstate (Lake Placid, N.Y.). A generic substrate, DiFMUP (6,8-difluoro-4-methylumbelliferyl phosphate) was purchased from Invitrogen (Carlsbad, Calif.). All assays were performed in 50 mM HEPES containing 1 mM DTT and 0.1% BSA, pH 7.4 with the following modifications or additions: SHP1, PTPMEG2, PTPβ, and YopH (10 mM MgCl₂); PP1α and PP2A (10 mM MnCl₂); HePTP, VHR, CD45, TC-PTP, SHP-2, LMPTPA (pH 4.5) and LMPTPB (pH 4.5); PTPMEG-1 (4.8 mM MgCl₂ and 3.2 mM MnCl₂); PTP-1B and DUSP22 (25 mM HEPES, 50 mM NaCl, 5 mM DTT and 2.5 mM EDTA). Compound (10 μM) was added to 15 μl enzyme and incubated for 10 minutes followed by 10 μL DiFMUP at a final concentration of 100 μM. The 384 well plate was incubated at room temperature for 60 minutes and then read in an Analyst (MDC using excitation 360 nm; emission 450 nm). The effect of the compound was compared to control wells containing DMSO.

To measure CD45 phosphatase activity in protein lysates, equal concentration of total protein (200 μg) from untreated or macrophages treated with PMOs (8 μM, 72 hours treatment) were first pre-cleared with protein G sepharose beads and then immunoprecipitated overnight with either the non-specific monoclonal antibody or CD45 specific (clone 30-F11, BD Biosciences) antibody in the presence of protein G beads. After washing, the beads were incubated with 100 μM of DiFMUP substrate in 100 μl of assay buffer for 1 hour. Supernatant was transferred into 96-well plates and fluorescence intensity was measured at excitation 358 nm and emission 455 nm. The experiments were repeated independently at least three times. The results are given as averages with standard deviation.

### Example 3

### Flow Cytometry

Antibodies used for FACS analysis were purchased from BD Pharmingen (San Diego, Calif.), unless otherwise noted. Antibodies used were directly conjugated to FITC, PE, APC, PerCP, or PECy5. Clones used in these studies included CD45 (30-F11), CD3 (17A2), CD4 (RM4-5), CD8 (53-6.7), CD11b (M1/70), CD11c (N418, eBioscience), CD19 (1D3), NK1.1 (PK136, eBioscience), MHC I (28-14-8), MHC II (M5/114.15.2), CD44 (IM7) and Ly6G (1A8). Cells (1×10⁶) were resuspended in Fc block (anti CD16/CD32 antibody diluted in RPMI medium containing 10% FBS), incubated on ice for 30 minutes, centrifuged and stained with appropriate combinations of labeled antibodies. After incubation on ice for 60 minutes, cells were washed and resuspended in 3.7% formaldehyde. FACS analysis was performed using a FACSCalibur flow cytometer (BD Biosciences). Data was analyzed using FlowJo software (Tree Star, Inc; Ashland, Oreg.).

### Example 4

### B. anthracis Challenge Studies

To test the effects of compounds on cell viability following B. anthracis infection, J774A.1 cells (6×10⁵) were pretreated with DMSO control or compound (10 μM). After 1 hour cells were infected with Sterne B. anthracis spores (5 MOI). After incubation for 4 hours at 37° C., bacterial growth was inhibited by the addition of antibiotics, penicillin (100 IU) and streptomycin (100 μg/ml). To determine cell viability SYTOX green nucleic acid stain (1 μM, Molecular probes) that is impermeant to live cells was added and incubated for 15 minutes at 37° C. The cells were centrifuged at 2000 rpm for 2 minutes and then washed two times with complete medium containing antibiotics. The cells were fixed with 1% formaldehyde for 15 minutes and then analyzed by flow cytometry.

To test the effects of CD45 knock-down on cell viability following B. anthracis infection, J774A.1 cells (6×10⁵) were either left untreated or treated with CD45 PMO or SC PMO. After 72 hours cells were harvested and infected with the Sterne B. anthracis spores (5 MOI). After incubation for 4 hours at 37° C., cell viability was measured by the uptake of SYTOX green dye (as described above).

### Example 5

### Immunoblot Analysis

J774A.1 cells (about 1×10⁶) seeded in a 6 well plate were either left untreated or incubated with CD45 PMO or SC PMO for 72 hours. Cells were harvested and lysed in buffer containing 50 mM Tris-HCl, pH 7.4, 150 mM NaCl, 2 mM EDTA, 1% Triton X-100, and protease inhibitor cocktail (Sigma). The cell lysates were incubated for 30 minutes on ice and then centrifuged for 30 minutes at 14,000 rpm. Cell extracts (30 μg) were electrophoresed on SDS-PAGE and then subjected to western blotting. A CD45 specific mouse monoclonal antibody (clone 69, BD Pharmingen) was used to detect the immunoreactive proteins that were visualized by Enhanced Chemiluminescence (ECL).

To determine the MEK cleavage pattern, J774A.1 cells were treated with the 8 μM CD45 PMO or 8 μM SC PMO for 72 hours. Cells were harvested and then infected with Sterne B. anthracis spores (5 MOI). After incubation for 4 hours, cells were washed with phosphate buffered saline (PBS), lysed and electrophoresed as described above. Western blots were probed with MEK1″NT″ antibody (Upstate Biotechnology) or GAPDH for uniform protein loading and visualized by ECL.

### Example 6

### Animal Studies

For CD45 studies, eight to ten week old mice were used and included both males and females. For in vivo B. anthracis studies, C57BL/6 wild type mice (controls) or CD45^{62%} or mice expressing different levels of CD45 were challenged via intraperitoneal (i.p.) route with 500 cfu of Ames strain of B. anthracis. The mice were monitored for one month post challenge. Mice that survived were either rechallenged or blood was collected to measure anti PA antibody levels in the serum by ELISA and tissues harvested for pathology.

For in vivo Ebola studies, mice were challenged via intraperitoneal (ip) route with 1000 pfu of mouse adapted Ebola Zaire virus. Mice that survived EBOV challenge were either rechallenged, or had their blood collected to measure anti-EBOV antibody levels in the serum by ELISA and tissues were harvested for pathology, T cell responses and viral titer. See Warfield et al. (2006) PLoS Pathog. 2, e1 and Moe, J. B. et al. (1981) J. Clin. Microbiol. 13:791-793, which are herein incorporated by reference.

### Example 7

### Phagocytosis and Spore Viability

To enumerate the spores ingested by macrophages, thioglycolate elicited peritoneal macrophages from 100%, 62% and 0% mice were infected with 5 MOI of GFP-Sterne spores and plate centrifuged to synchronize the infection. After 30 minutes, non-permeabilized cells were incubated with a mix of antibodies specific for B. anthracis spore exosporium (to label extracellular spores) and bacillus polysaccharide (to label extracellular vegetative bacilli) (kindly provided by T. Abshire and J. Ezzel, USAMRIID), followed by a secondary incubation with antibody conjugated to Alexa-594 nm fluorophore. This method labels only those spores adhered to the outside surface of the macrophages. After fixation with formaldehyde, cells were stained with Hoechst dyes and images from nine sites/well were collected and analyzed using the Discovery-1 high-content screening system (Molecular devices, Downington, Pa.). Images were analyzed using the cell health module of MataXpress imaging analysis software. Total cell count was based on the number of Hoechst-stained cell nuclei, while colocalization with red (anti-spore and anti-bacterial antibody) and GFP-Sterne spores was scored as spores being on the outside of the cell and colocalization with GFP-Sterne spores are ingested spores. Thioglycolate elicited peritoneal macrophages purified by plastic adherence were infected with Sterne B. anthracis spores at an MOI of 5. After 30 minutes, cell pellets collected by centrifugation were lysed in sterile water, serially diluted and aliquots were plated onto solid LB agar medium plates, which were then incubated at 37° C. for 16 hours. Colony forming units (CFU) were counted and data are represented as CFU/ml. Experiments were performed in duplicate and repeated three independent times.

### Example 8

### Immunohistochemical Staining

To detect the presence of bacilli in infected tissues, the EnVision systems (Dako) was used. Briefly, tissue sections were deparaffinized, blocked in methanol/H₂O₂ solution for 30 minutes at room temperature (RT), rinsed with water, pretreated with Tris-EDTA, pH 9.0 at 97° C. for 30 minutes and then blocked with the mouse IgG blocking buffer (Vector Lab, 1: 20). The tissues were then incubated with mouse anti-capsule antibody (#593) or mouse IgG as negative control serum for 30 minutes at room temperature. After rinsing three times with PBS, peroxidase labeled polymer conjugated to goat anti-mouse immunoglobulins was added and incubated for 30 minutes. After rinsing with PBS, substrate-chromogen solution was added, incubated for 5 minutes, rinsed with PBS, stained with hematoxylin (Dako, Carpinteria, Calif.), dehydrated and tissues were mounted with Permount.

To detect the presence of EBOV in infected organs, the tissue sections were deparaffinized, blocked in methanol/H₂O₂ solution for 30 minutes at room temperature, rinsed with water and pretreated with freshly diluted Proteinase K solution (20 μg/ml) for 15 minutes at room temperature (RT). After blocking with the CAS block containing 5% goat serum, the sections were incubated with Rabbit anti-Ebola (994) antibody for 60 minutes at room temperature. After rinsing three times with PBS, peroxidase labeled polymer conjugated to goat anti-rabbit immunoglobulins was added and incubated for 30 minutes. After rinsing with PBS, substrate-chromogen solution was added, incubated for 5 minutes, rinsed with PBS, stained with hematoxylin (Dako, Carpinteria, Calif.), dehydrated and tissues were mounted with Permount.

Apoptosis in tissues was detected by TUNEL using the APOPTAG® Plus Peroxidase In Situ Apoptosis Kit (Chemicon, Temecula, Calif.) as described above, but with the following changes. After proteinase K treatment and washing, the tissues were incubated with terminal deoxynucleotidyl transferase (TdT) for 60 minutes at 37° C. The reaction was stopped by adding stop/wash buffer and incubating for 10 minutes at room temperature. After washing three times with PBS, the tissues were incubated with anti-digoxigenin-peroxidase for 30 minutes at room temperature. Color development was visualized after incubation with the DAB substrate. Slides were counterstained in methyl green.

### Example 9

### Gene Expression Studies and Microarray Analysis

Splenocytes isolated from spleen of wild type (100%) and heterozygous (62%) mice at day 0, 1, 3 and 5 were resuspended in 2 ml TRIzol solution. The samples were stored at −70° C. until the RNA was purified. Total cellular RNA was isolated as per the manufacturer's specifications. The quality and concentration of the RNA were determined by measuring the absorbance at 260 and 280 nm. RNA integrity was confirmed by an Ailent 2100 Bioanalyzer (Agilent technologies, Palo Alto, Calif., USA). The mouse genome 430 2.0 array (Affymetrix, Inc.), which consists of over 39,000 genes in a single array, was used. Affymetrix CEL files were preprocessed using GCRMA in R/Bioconductor. See Gentleman, R. C. et al. (2004) Genome Biol. 5:R80, which is herein incorporated by reference. Median log2 expression values within each sample type/time point with coefficient of variation (CV) >0.4 were chosen for agglomerative hierarchical clustering. Genes on the X or Y chromosome were removed and the probeset with the largest CV was chosen for genes with >1 probeset. Cluster stability was assessed with clusterStab to suggest cluster boundaries. See Smolkin & Ghosh (2003) BMC Bioinformatics 4:36, which is herein incorporated by reference.

As disclosed herein, mice expressing a range of reduced CD45 levels were protected from infection by B. anthracis and EBOV. In contrast, wild type mice, mice with inactive CD45 phosphatase activity, and CD45 knockout mice were not protected against infection by B. anthracis and EBOV. Reduced CD45 expression had no effect on bacterial and viral uptake, viral replication or mouse humoral responses. While reduced apoptosis was observed in expressing intermediate levels of CD45 infected with B. anthracis, survival of mice infected with EBOV was independent of the apoptotic pathway. Although the precise downstream signaling pathways involved appear to be pathogen-specific, the results of the experiments disclosed herein suggest that modulation of CD45 expression levels can elicit dynamic host immunity via accelerated immune cell homeostasis and cell trafficking in response to infection by diverse pathogens, such as B. anthracis and EBOV.

Therefore, the present invention provides methods for preventing, reducing or inhibiting the expression level of a protein tyrosine phosphatase (PTP), such as CD45, or activity of the PTP in a cell or a subject. The present invention also provides methods for preventing, inhibiting or treating an infection in a cell or a subject which comprises reducing or inhibiting the expression level of a protein tyrosine phosphatase (PTP), such as CD45, or activity of the PTP in the cell or the subject. The present invention also provides methods for immunizing a subject or enhancing a subject's immune response against an infection which comprises reducing or inhibiting the expression level of a protein tyrosine phosphatase (PTP), such as CD45, or activity of the PTP in the subject and administering an antigenic or immunological amount of the biological agent. The present invention provides methods for preventing, reducing or inhibiting the susceptibility of a cell or a subject to an infection and subsequent pathogenesis and morbidity caused by a biological agent which comprises reducing or inhibiting the expression level of a protein tyrosine phosphatase (PTP), such as CD45, or activity of the PTP in the cell or the subject.

The present invention also provides methods for increasing, improving or enhancing
clearance of a biological agent in a cell or a subject,
an immunological response to a biological agent by a cell or a subject,
the viability of a cell or a subject exposed to or infected with a biological agent, or
the number of macrophages and dendritic cells in a subject infected with a biological agent,
   which comprises reducing or inhibiting the expression level of a protein tyrosine phosphatase (PTP), such as CD45, or activity of the PTP in the cell or the subject.

The present invention also provides methods for preventing, reducing, or inhibiting apoptosis caused by or resulting from a biological agent in a cell or a subject, which comprises reducing or inhibiting the expression level of a protein tyrosine phosphatase (PTP), such as CD45, or activity of the PTP in the cell or the subject.

In some embodiments, the expression level of a protein tyrosine phosphatase (PTP), such as CD45, or activity of the PTP in a cell or a subject may be reduced or inhibited by administering an effective amount of a compound of the present invention or an antisense oligonucleotide such a CD45 PMO to the cell or the subject or using recombinant methods known in the art to knock-out or knock-down the gene encoding CD45 in the cell or the subject.

Prior studies have found that regulation of CD45 function can be modulated by factors such as phosphatase substrate binding, dimerization, subcellular localization, interacting proteins and specific intracellular inhibitors. See Hermiston, M. L. et al. (2003) Ann. Rev. Immunol. 21:107-137, which is herein incorporated by reference. Thus, following infection by a pathogen, such as B. anthracis or EBOV, modulation of one or more of these factors may result in immunity or resistance to the pathogen or have an effect on pathogenesis and morbidity. Therefore, compounds and compositions which prevent, reduce or inhibit expression levels of CD45 in a cell or a subject are contemplated herein and may be used in accordance with the teachings and invention(s) disclosed herein.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. (canceled)

2. The method of claim 4, wherein the compound is NSC 148596, NSC 135880, NSC 95397, NSC 270011 or NSC 270012.

3. (canceled)

4. A method of preventing, inhibiting or treating an infection by a bacterium or a virus or apoptosis caused by the infection in a macrophage or a subject which comprises preventing, reducing or inhibiting an amount of protein tyrosine phosphatase receptor type C (CD45) expressed or activity of CD45 in the macrophage or the subject by administering to the macrophage or the subject an effective amount of
a compound having one of the following structural formulas 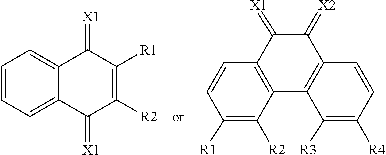
wherein R1, R2, R3, and R4 are each independently selected from the group consisting of hydrogen, amino, amidine, guanidine, carboxyl, alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, an amide possessing an alkyl substituent, each of which may be substituted or unsubstituted, or X—R wherein X is O, S, or N and R is selected from the group consisting of hydrogen, amino, amidine, guanidine, carboxyl, alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, or an amide possessing an alkyl substituent, each of which may be substituted or unsubstituted; and
X1 and X2 are each independently selected from the group consisting of O, S, or N which may be optionally substituted.

5-6. (canceled)

7. The method of claim 4, wherein the bacterium is Bacillus anthracis.

8. The method of claim 4, wherein the virus belongs to the family Filoviridae.

9. The method of claim 4, wherein the virus is an Ebolavirus or a Marburgvirus.

10. A method of immunizing a subject or enhancing a subject's immune response against an infection by a bacterium or a virus which comprises preventing, reducing or inhibiting an amount of protein tyrosine phosphatase receptor type C (CD45) expressed or activity of CD45 in the subject by administering to the subject an effective amount of
a compound having one of the following structural formulas 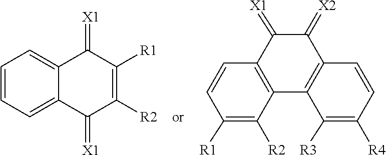
wherein R1, R2, R3, and R4 are each independently selected from the group consisting of hydrogen, amino, amidine, guanidine, carboxyl, alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, an amide possessing an alkyl substituent, each of which may be substituted or unsubstituted, or X—R wherein X is O, S, or N and R is selected from the group consisting of hydrogen, amino, amidine, guanidine, carboxyl, alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, or an amide possessing an alkyl substituent, each of which may be substituted or unsubstituted; and
X1 and X2 are each independently selected from the group consisting of O, S, or N which may be optionally substituted.

11. A method of preventing, reducing or inhibiting the susceptibility of a cell or a subject to an infection by a bacterium or a virus or subsequent pathogenesis and morbidity due to the infection which comprises preventing, reducing or inhibiting an amount of protein tyrosine phosphatase receptor type C (CD45) expressed or activity of CD45 in the subject or in macrophages of the subject by administering to the subject or the macrophages of the subject an effective amount of
a compound having one of the following structural formulas 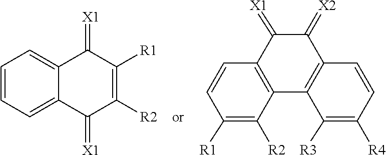
wherein R1, R2, R3, and R4 are each independently selected from the group consisting of hydrogen, amino, amidine, guanidine, carboxyl, alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, an amide possessing an alkyl substituent, each of which may be substituted or unsubstituted, or X—R wherein X is O, S, or N and R is selected from the group consisting of hydrogen, amino, amidine, guanidine, carboxyl, alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, or an amide possessing an alkyl substituent, each of which may be substituted or unsubstituted; and
X1 and X2 are each independently selected from the group consisting of O, S, or N which may be optionally substituted.

12-14. (canceled)

15. A method of preventing, inhibiting or treating an infection caused by Bacillus anthracis or a virus belonging to the family Filoviridae in a macrophage or a subject which comprises preventing, reducing or inhibiting an amount of protein tyrosine phosphatase receptor type C (CD45) expressed or activity of CD45 in the macrophage or the subject as compared to a wild-type control by administering to the macrophage or the subject an effective amount of
a compound having one of the following structural formulas 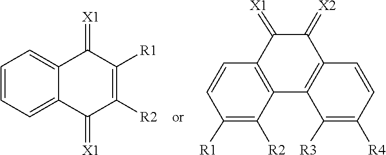
wherein R1, R2, R3, and R4 are each independently selected from the group consisting of hydrogen, amino, amidine, guanidine, carboxyl, alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, an amide possessing an alkyl substituent, each of which may be substituted or unsubstituted, or X—R wherein X is O, S, or N and R is selected from the group consisting of hydrogen, amino, amidine, guanidine, carboxyl, alkyl, alkenyl, alkynyl, cycloalkyl, heterocycloalkyl, aryl, heteroaryl, alkoxy, aryoxy, cycloalkoxy, heteroaryloxy, alkoxycarbonyl, alkylamino, carbamoyl, alkylaminocarbonyl, alkylsulfhydryl, alkylhydroxymate, or an amide possessing an alkyl substituent, each of which may be substituted or unsubstituted; and
X1 and X2 are each independently selected from the group consisting of O, S, or N which may be optionally substituted.

16. The method of claim 10, wherein the macrophages of the subject exhibit a reduction in expression or activity of protein tyrosine phosphatase receptor type C (CD45).

17. The method of claim 10, wherein the compound is NSC 148596, NSC 135880, NSC 95397, NSC 270011 or NSC 270012.

18. The method of claim 10, wherein the bacterium is Bacillus anthracis.

19. The method of claim 10, wherein the virus belongs to the family Filoviridae.

20. The method of claim 10, wherein the virus is an Ebolavirus or a Marburgvirus.

21. The method of claim 11, wherein the compound is NSC 148596, NSC 135880, NSC 95397, NSC 270011 or NSC 270012.

22. The method of claim 11, wherein the bacterium is Bacillus anthracis.

23. The method of claim 11, wherein the virus belongs to the family Filoviridae.

24. The method of claim 11, wherein the virus is an Ebolavirus or a Marburgvirus.

25. The method of claim 15, wherein the compound is NSC 148596, NSC 135880, NSC 95397, NSC 270011 or NSC 270012.
